(19) United States
(12) Patent Application Publication
(10) Pub. No.: US 2023/0100000 A1
(43) Pub. Date: Mar. 30, 2023

(54) Immune cell expressing adapter chimeric antigen receptor for sensing soluble antigens

(71) Applicant: Miltenyi Biotec B.V. & Co. KG, Bergisch Gladbach (DE)

(72) Inventors: Niels Werchau, Bergisch Gladbach (DE); Bettina Kotter, Bergisch Gladbach (DE); Joerg Mittelstaet, Bergisch Gladbach (DE); Andrew Kaiser, Bergisch Gladbach (DE)

(73) Assignee: Miltenyi Biotec B.V. & Co. KG, Bergisch Gladbach (DE)

(21) Appl. No.: 17/797,419

(22) PCT Filed: Feb. 3, 2021
(86) PCT No.: PCT/EP2021/052482

§ 371 Date: Aug. 3, 2022

(30) Foreign Application Priority Data
Feb. 4, 2020 (EP) 20155377.3

(51) Int. Cl. A61K 35/17; C12N 5/0783

(52) U.S. Cl. CPC A61K 35/17; C12N 5/0636; A61K 2039/5156

(57) ABSTRACT

The present invention provides a composition comprising a) an immune cell comprising a polynucleotide encoding an adapter CAR specific for an adapter, b) the adapter specific for a soluble antigen, and c) the soluble antigen. In an embodiment of the invention, the immune cell expressing said adapter CAR comprises in addition a nucleic acid comprising an inducible promoter operably linked to a nucleic acid encoding an effector such as a synthetic.

### FIELD OF THE INVENTION

The present invention generally relates to the field of chimeric antigen receptors (CARs) expressed on immune cells, in particular to adapter CARs (anti-tag CARs) that bind via an adapter to soluble antigens.

### BACKGROUND OF THE INVENTION

Chimeric antigen receptor (CAR) T cell therapy has shown remarkable success for the treatment of a series of hematological malignancies such as acute lymphoblastic leukemia. It is based on genetically modifying T cells to express a tumor-specific CAR receptor that induces T cell activation, proliferation and cytolytic activity within the patient when the tumor antigen has been recognized. CARs specifically engage the target via the antigen recognition moiety, e.g. of a single chain variable fragment (scFv) derived from antibodies or a Fab fragment. But, application of CAR immune therapy to solid tumors has been relatively limited and has thus far demonstrated limited success. This limited success of treatment of solid tumors by CAR immune therapy may be caused among others by immune cell intrinsic features such as immune (T) cell exhaustion, as well as active cancer immunosuppressive mechanisms, hostile tumor microenvironment (TME), presence of various immunosuppressive cells, and lack of suited surface antigens.

In WO2019199689A1 engineered immune cells are disclosed that comprise a single viral vector comprising both a first polynucleotide comprising a constitutive promoter operably linked to a nucleic acid encoding at least one transgene, e.g. a CAR; and a second polynucleotide comprising an inducible promoter operably linked to a nucleic acid encoding an effector. This allows e.g. an in-situ-expression of an effector such as IL-6 or another inflammatory signaling axis in a tumor microenvironment after binding of the CAR that has specificity to the tumor antigen expressed on the surface of the solid tumor.

Chang et al. (2018, Nat Chem Biol. 14:317-324) disclosed that CAR-T cells can be engineered to respond robustly to soluble ligands, provided that the ligands are capable of mediating CAR dimerization. They suggested a few features that may be important for CARs to be triggered by a soluble ligand. First, the soluble ligand must be able to bring together two or more CARs. Second, the CAR-ligand binding affinity must be strong enough to withstand the tensile force necessary for CAR activation. Third, both the soluble ligand and the CARs involved must be mechanically rigid enough to transmit tensile force to the CARs' intracellular signaling domains.

“Universal” CAR systems (or adapter CAR (adCAR) systems) that indirectly bind to target cells via adapters are described e.g. in WO2012082841A2, WO2013044225A1 and WO2016030414A1. Therein adapters such as tagged antibodies that bind to surface-bound antigens expressed on the target cell and tag-specific CARs that are expressed by immune cells are disclosed, wherein the tag may be either artificial (such as FITC) and potentially immunogenic or an endogenous molecule which may compete with the natural counterparts to the CAR binding.

There is a need in the art for an improved or alternative adapter CAR system. In addition, there is a need in the art to use an improved or alternative adapter CAR system in a clinical environment, e.g. to sense soluble antigens of a tumor microenvironment in a subject suffering from said tumor, and allow the immune cell expressing the adapter CAR to execute subsequently cytolytic activities against this tumor.

### SUMMARY OF THE INVENTION

It was surprisingly found that an immune cell expressing an adapter CAR that specifically binds to an adapter that binds specifically to a soluble antigen such as a cytokine that may be present e.g. in a tumor microenvironment (TME) can be activated, when said adapter CAR binds the adapter that is bound to the soluble antigen. The essential requirement for the activation of the immune cell expressing the adapter CAR is the soluble antigen mediated dimerization of the adapter CAR. This finding is unexpected as the use of an additional, non-covalent bound spacer molecule, i.e. the adapter, is inserted between the antigen and the CAR expressed on the surface of the immune cell, shifting the CAR system to a more instable system as compared to a CAR that directly binds the soluble antigen. Nonetheless and unexpectedly, this adapter CAR system is mechanically rigid enough to transmit tensile force to the CARs' intracellular signaling domains when the adapter and the soluble antigen fulfill the criteria as disclosed herein.

The present invention provides a composition comprising a) an immune cell comprising a polynucleotide encoding an adapter CAR specific for an adapter, b) the adapter specific for a soluble antigen, and c) the soluble antigen. In an embodiment of the invention, the immune cell expressing said adapter CAR comprises in addition a nucleic acid comprising an inducible promoter operably linked to a nucleic acid encoding an effector such as a synthetic transcription factor, a cytokine or a second CAR.

### BRIEF DESCRIPTION OF THE DRAWINGS

FIG. 1: Sensing soluble antigens with anti-tag CAR T cells.

Schematic representation of a T cell expressing a CAR on the cell surface. The CAR consists of an intracellular signaling domain, a transmembrane domain and a scFv for binging the tagged polypeptide. The tagged polypeptide mediates the binding of the soluble antigen and CAR and serves as an adapter. The adapter molecule and the soluble target antigen are in solution. The binding of the adapter molecule to the antigen and the subsequent binding of the antigen/adapter complex by the CAR expressed on the cell surface results in receptor multimerization on the cell surface and activation of cell signaling pathways. The soluble antigen can be sensed by the CAR only in the presence of the adapter molecule. The structural properties of the soluble antigen define the setting of the antigen binding domain for the adapter molecules:
a) The soluble antigen is providing an antigen A and B, according to this the tagged polypeptide contains an antigen binding domain for epitope A and B.
b) The soluble antigen is providing an antigen A and B, according to this the first tagged polypeptide contains an antigen binding domain for epitope A and the second tagged polypeptide contains an antigen binding domain for epitope B.
c) The soluble antigen is providing two times an epitope E, according to this the tagged polypeptide contains an antigen binding domain for epitope E.

FIG. 2: Application for sensing soluble factors via anti-tag CAR T cells.

CAR T cells are typically designed to respond to surface-bound antigens and not to soluble antigens in the TME, which would be a key aspect in order to expand the CAR T cells therapy to solid tumors.

The system allows the sensing of multiple soluble factors of the TME with just one anti-tag CAR expressed on the surface of a T cell, just by exchanging the tagged polypeptide. This means a reduction in manufacturing time and cost. Furthermore it allows more flexibility in responding to the tumor related TME.

The system allows the recognition of the TME in a local, dose dependent and time controlled manner. Thereby the TME triggers the activation of anti-tag CAR T cells which results for instance in the secretion of cytokines. The local activation of T cells by TME molecules would increase the safety profile as activation of the CAR T cells is restricted to the tumor area.

Thus the anti-tag CAR T cells prime the microenvironment by inducing a local inflammation and can enhance the subsequent cytotoxic CAR T cell response either against tumor associated antigens or TME related target cells.

FIG. 3: Activation of anti-tag CAR T cells via homodimeric soluble LAP requires at least one tagged adapter molecule.

The activation of anti-tag CAR T cells via soluble molecules requires the formation of a complex between antigen and adapter molecule. Homodimeric recombinant human LAP was used and therefore one adapter molecule containing the binding site for this epitope leads to anti-tag CAR T cell activation.

T cells of three healthy independent donors were transduced with LV particles encoding for an anti-biotin CAR. Transduced T cells were co-cultured with 125 ng/mL recombinant human LAP (grey bars) or without recombinant human LAP (white bars). Two different anti human LAP antibody clones labeled with biotin were used as adapter molecules (clone 1:CH6-17E5.1 and clone2: TW4-2F8). The adapters were either used at a 1:1 ratio with a concentration of 10 ng/mL for each adapter, or the adapters were applied individually.

After 24 h the mean of CD69 (a) and CD25 (b) amongst LNGFR positive cells was analyzed via flow cytometry. Values were normalized to the corresponding mean of anti-tag CAR T cells without the two different anti-human LAP antibody clones labeled with biotin and recombinant human LAP.

FIG. 4: Activation of anti-tag CAR T cells via soluble antigens depends on the concentration of the adapter molecule.

T cells of three healthy independent donors were transduced with LV particles encoding for an anti-biotin CAR. Transduced T cells were co-cultured with 250 ng/mL of recombinant human LAP and two different anti human LAP antibody clones labeled with biotin (clone 1:CH6-17E5.1 and clone2: TW4-2F8). A 10-fold serial dilution of two different anti human LAP antibody clones labeled with biotin was made, ranging from 100 ng/mL to 0.1 ng/mL. After 24 h the mean of CD69 (a) and CD25 (b) amongst LNGFR positive cells was analyzed via flow cytometry. Values were normalized to the corresponding mean of anti-tag CAR T cells without the two different anti-human LAP antibody clones labeled with biotin and recombinant human LAP.

FIG. 5: Activation of anti-tag CAR T cells via soluble antigens depends on the concentration of the soluble ligand.

T cells of three healthy independent donors were transduced with LV particles encoding for an anti-biotin CAR. Transduced T cells were co-cultured with 100 ng/mL of two different anti human LAP antibody clones labeled with biotin (clone 1:CH6-17E5.1 and clone2: TW4-2F8) and recombinant human LAP.

A 2-fold serial dilution of recombinant human LAP was made. Ranging from 500 ng/mL to 7.81 ng/mL. After 24 h the mean of CD69 (a) and CD25 (b) amongst LNGFR positive cells was analyzed via flow cytometry. Values were normalized to the corresponding mean of anti-tag CAR T cells without the two different anti human LAP antibody clones labeled with biotin and recombinant human LAP.

FIG. 6: Anti-tag CAR T cells secrete cytokines upon stimulation via soluble antigens.

Polyfunctional anti-tag CAR T cells activated by soluble antigens are characterized by the secretion of cytokines.

T cells of two independent healthy donors were transduced with LV particles encoding for an anti-biotin CAR. Transduced T cells were co-cultured with 10 ng/mL of two different anti human LAP antibody clones labeled with biotin (clone1:CH6-17E5.1 and clone2: TW4-2F8) and 125 ng/mL recombinant human LAP. After 24 h, supernatant of the co-culture was analyzed via MACS Plex.
a) GM-CSF secretion after the stimulation of CAR T cells with soluble antigen in the presence or absence of LAP.
b) INF-γ secretion after the stimulation of CART cells with soluble antigen in the presence or absence of LAP.
c) IL-2 secretion after the stimulation of CAR T cells with soluble antigen in the presence or absence of LAP.
d) TNF-α secretion after the stimulation of CAR T cells with soluble antigen in the presence or absence of LAP.

FIG. 7: Soluble antigens recognized by anti-tag CAR T cells can be used to induce transgene expression.

The specific activation of anti-tag CAR T cells via the sensing of soluble antigens can be used to specifically induce the expression of a gene of interest.
a) CAR T cells were modified in order to express GFP under the control of a NFAT/AP-1 promoter sequence from human IL-2 sequence. A four times repeat of a NFAT/AP-1 DNA binding sequence is located 5′ of the gene of interest e.g. GFP.
b) Soluble antigens recognized by CAR T cells can be used to induce transgene expression. T cells of two independent healthy donors were transduced with LV particles encoding for an anti-biotin CAR. Anti-tag CAR T cells were incubated with 250 ng/ml or 125 ng/mL recombinant human LAP and 10 ng/mL of an anti-human LAP antibody labeled with biotin (clone 1:CH6-17E5.1). Cells were incubated for a total of 4 days. Transgene expression was quantified via flow cytometry.

### DETAILED DESCRIPTION OF THE INVENTION

In a first aspect, the invention provides a composition comprising

A)

a) an immune cell comprising a polynucleotide comprising a nucleic acid encoding a chimeric antigen receptor (CAR) comprising

i) an antigen binding domain specific for a tag of a tagged polypeptide or for two tagged polypeptides having the same tag

ii) a transmembrane domain

iii) an intracellular signaling domain

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen,

c) said soluble antigen, wherein said soluble antigen comprises said epitope A and said epitope B,

or

B)

a) an immune cell comprising a polynucleotide comprising a nucleic acid encoding a chimeric antigen receptor (CAR) comprising

i) an antigen binding domain specific for a tag of a tagged polypeptide

ii) a transmembrane domain

iii) an intracellular signaling domain

b) said tagged polypeptide, wherein said tagged polypeptide comprises an antigen binding domain specific for an epitope E of a soluble antigen

c) said soluble antigen, wherein said soluble antigen comprises at least two times said epitope E.

Said intracellular (cytoplasmic) signaling domain may comprise at least one primary cytoplasmic signaling domain comprising an immunoreceptor tyrosine-based activation motif (ITAM) and/or at least one co-stimulatory signaling domain.

Said primary cytoplasmic signaling domain of said first CAR may be CD3zeta.

Said at least one co-stimulatory domain of said first CAR, may be selected from the group consisting of ICOS, CD154, CD5, CD2, CD46, HVEM, CD8, CD97, TNFRSF18, CD30, SLAM, DAP10, CD64, CD16, CD89, MyD88, KIR-2DS, KIR-3DS, NKp30, NKp44, NKp46, NKG2D, ICAM, CD27, OX40, 4-1BB, and CD28.

Said composition(s) as disclosed herein, wherein binding of the antigen binding domain of said CAR to said tagged polypeptide that has bound thereto said soluble antigen activates said immune cell.

Said immune cell may be a T cell or a NK cell.

Said composition(s) as disclosed herein, wherein said tagged polypeptide(s) that bind(s) to said soluble antigen may be an antibody or antigen binding fragment thereof.

Said tag may be a hapten.

Said tag may be selected from the group consisting of dextran, biotin, fluorescein isothiocyanate (FITC), phycoerythrin (PE), thiamin, peptides such as c-Myc-tag, Strep-Tag, Flag-Tag, Polyhistidine-tag or proteins such as streptavidin. Preferentially, the tag may be biotin or a derivate thereof.

The composition(s) as disclosed herein, wherein said soluble antigen of A) may be a monomeric antigen comprising said epitope A and said epitope B, and/or wherein said soluble antigen of B) may be a homo-dimeric or a multimeric antigen comprising at least two times said epitope E.

The compositions of A) and/or B) as disclosed herein, wherein crosslinking of said soluble antigen and said tagged polypeptide(s) allows dimerization of said CAR.

The composition(s) as disclosed herein, wherein said soluble antigen of A) and/or said soluble antigen B) may be

I) a soluble antigen of a tumor microenvironment (TME), or

II) a soluble antigen specifically associated with an autoimmune disease, or

III) a soluble antigen specifically associated with an allergic disease, or

IV) a soluble antigen specifically associated with an infectious disease, or

V) a soluble antigen specifically associated with graft rejection in a subject

Said tumor microenvironment (TME) may be a TME of a subject suffering from cancer.

Said autoimmune disease may be an autoimmune disease of a subject suffering from said autoimmune disease.

Said allergic disease may be an allergic disease of a subject suffering from allergy.

Said infectious disease may be an infectious disease of a subject suffering from infection.

Said cancer/tumor may be a solid tumor.

Said solid tumor may be adrenal cancer, anal cancer, bile duct cancer, bladder cancer, bone cancer, brain/CNS tumors in children or adults, breast cancer, cervical cancer, colon/rectum cancer, endometrial cancer, esophagus cancer, ewing family of tumors, eye cancer, gallbladder cancer, gastrointestinal carcinoid tumors, gastrointestinal stromal tumor (GIST), gestation trophoblastic disease, hodgkin disease, kaposi sarcoma, kidney cancer, laryngeal and hypopharyngeal cancer, leukemia, acute lymphocytic leuckemia, acute myeloid leukemia, chronic lymphocytic leukemia, chronic myeloid leukemia, chronic myelomonocytic leukemia, liver cancer, lung cancer, non-small cell lung cancer, small cell lung cancer, lung carcinoid tumor, lymphoma, malignant mesothelioma, multiple myeloma, myelodysplastic syndrome, nasal cavity and paranasal sinum cancer, nasopharyngeal cancer, neuroblastoma, non-hodgkin lymphoma, oral cavity or oropharyngeal cancer, osteosarcoa, ovarian cancer, pancreatic cancer, penile cancer, pituitary tumors, prostate cancer, retinoblastoma, rhabdomyosarcoma, salivary gland cancer, skin cancer, sarcoma, basal skin cancer, squamous cell skin cancer, melanoma, merkel cell skin cancer, small intestine cancer, stomach cancer, testicular cancer, thymus cancer, thyroid cancer, uterine sarcoma, vaginal cancer, vulvar cancer, Waldenstrom macroglobulinemia, or wilms tumor

Autoimmune diseases are a condition arising from autoimmunity resulting in pathologies that can affect multiple different organ systems. Examples include Behcet's disease, Juvenile idiopathic arthritis, Type 1 diabetes, Rheumatoid arthritis, Wegener Granulomatosis, Systemic lupus erythematosus, Systemic sclerosis, Crohn's disease, Graves' disease, Hashimoto thyreoiditis, Goodpasture syndrome, pernicieuse anemia, Primary biliary cholangitis, Myasthenia gravis, Dermato polymyositis, Vasculitis, Mixed connective tissue disease, Scleroderma, Multiple sclerosis, Psoriasis, Ulcerative colitis and Uvetis.

Therefore, said autoimmune disease may be e.g. Behcet's disease, Juvenile idiopathic arthritis, Type 1 diabetes, Rheumatoid arthritis, Wegener Granulomatosis, Systemic lupus erythematosus, Systemic sclerosis , Crohn's disease, Graves' disease, Hashimoto thyreoiditis, Goodpasture syndrome, pernicieuse anemia, Primary biliary cholangitis, Myasthenia gravis, Dermato polymyositis, Vasculitis, Mixed connective tissue disease, Scleroderma, Multiple sclerosis, Psoriasis, Ulcerative colitis or Uvetis.

Allergies, or allergic diseases, are a number of conditions caused by hypersensitivity of the immune system to typically harmless substances in the environment. These diseases include hay fever, food allergies, atopic dermatitis, allergic asthma, and anaphylaxis.

Infection is the invasion of an organism's body tissues by disease-causing agents, their multiplication, and the reaction of host tissues to the infectious agents and the toxins they produce. Infections are caused by infectious agents (pathogens) including: viruses, bacteria, fungi and parasites. Said infection may be an acute or a chronic infection.

“Graft rejection” in a subject is an immunologic destruction of transplanted tissues or organs between two members or strains of a species differing at the major histocompatibility complex for that species (i.e. HLA in man and H-2 in the mouse).

Said compositions of A) and/or B) as disclosed herein, wherein said a soluble antigen may be a cytokine, a chemokine, a shed surface receptor, extracellular matrix remodeling enzymes, a circulating tumor nucleic acid or tumor reactive antibody.

Said shed surface receptor may be a receptor that originally was present on a tumor cell but was shed by said tumor cell and circulates subsequently in the blood of the subject harboring said tumor cell.

Said circulating tumor nucleic acid may be a nucleic acid that originally was present in a tumor cell but was secreted by said tumor cell and circulates subsequently in the blood of the subject harboring said tumor cell.

Said tumor reactive antibody may be an antibody applied to a subject suffering from said tumor or an endogenous antibody secreted by a B cell of said subject that specifically bind to a tumor antigen or tumor associated antigen of said tumor (on said tumor cell).

Said extracellular matrix remodeling enzymes may be enzymes applied to a subject suffering from said tumor and influence the physical properties of the extracellular matrix of said tumor.

Said compositions of A) and/or B) as disclosed herein, wherein said soluble antigen specifically associated with the TME may be e.g. arginase, carcinoembryonic antigen, CCL11, CCL18, CCL2, CCL5, CD282, Circulating Tumor Nucleic Acids, CXCL10, FAP, GM-CSF, IFN-γ, IL-4, IL-6, IL-7 IL-8, IL-10, IL-11, IL-12, Il-13, IL-14, IL-15, IL-17, IL-23, IL-33, IL-1beta, IL-1Ra, INF, LAP, M-CSF, MMP12, MMP13, MMP7, NY-ESO-1 antibody, prostate-specific antigen, sCD106, sCD137, sCD152, sCD223, sCD25, sCD27, sCD253, sCD270, sCD273, sCD274, sCD279, sCD28, sCD30, sCD366, sCD40, sCD54, sCD80, sCD86, sGITR, TGFβ-1, TGFβ-2, TGFβ-3, TIMP1 or TNF-α, VEGF. The “s” in sCDx regularly stands for a soluble form of the respective CDx molecule.

Said compositions of A) and/or B) as disclosed herein, wherein said soluble antigen specifically associated with an autoimmune disease may be e.g. CCL2, CCL3, CCL4, CCL5, CSCL11, CXCL10, CXCL8, CXCL9, GM-CSF, IL-1, IL10, IL-11, IL-12, IL-15, IL-17, IL-18, IL-2, IL-21, IL-22, IL-23, IL-6, IL-7, IL-8, INF or TNF.

Said compositions of A) and/or B) as disclosed herein, wherein said soluble antigen specifically associated with an allergic disease may be allergen specific IgE, eotaxin, GM-CSF, IFN, IL-13, IL-21, IL-31, IL-4, IL-5, IL-9, MCP-1, MCP-3, MCP-4, MDC, sCD23, sCD93 or TNF.

Said compositions of A) and/or B) as disclosed herein, wherein said soluble antigen specifically associated with an infectious disease may be e.g. CRP, IL-15, IL-27, IL-6, IL-7, IL-8, INF, sCD14, sCD163, soluble urokinase-type plasminogen activator receptor or sTREM-1

Said compositions of A) and/or B) as disclosed herein, wherein said soluble antigen specifically associated with graft rejection may be e.g. CCL2, CCL4, CXCL10, CXCL11, IL-2, IL-4, IL-6, IL-15, IL-18, IL-23, IFN, sCD4, sCD8, TNF or XCL1.

Said composition of A), wherein said antigen binding domain specific for an epitope A and said antigen binding domain specific for an epitope B of said tagged polypeptide(s) may have a binding affinity to said epitope A and epitope B of said soluble antigen that may be at least 100 μM, at least 1 μM, at least 100 nM, at least 1 nM, at least 100 pM or at least 1 pM.

Said composition of B), wherein said antigen binding domain specific for an epitope E of said tagged polypeptide may have a binding affinity to said epitope E of said soluble antigen that may be at least 100 μM, at least 1 μM, at least 100 nM, at least 1 nM, at least 100 pM or at least 1 pM.

The affinity at which the tagged polypeptide binds to the CAR can vary, but generally the binding affinity may be in the range of 0.01 nM to 1 μM, preferentially in the range of 0.1-100 nM, or more preferentially in the range of 1-30 nM.

Said ranges of binding affinity of said antigen binding domain of said tagged polypeptide(s) to said epitope A and epitope B or to said epitope E of said soluble antigen as disclosed herein and said ranges of said affinities at which the tagged polypeptide binds to the CAR as disclosed herein may be combined.

Said compositions as disclosed herein, wherein the composition of A may comprise additionally

d) a tagged polypeptide or two tagged polypeptides, wherein said tagged polypeptide may comprise an antigen binding domain specific for an epitope C of a second soluble antigen and an antigen binding domain specific for an epitope D of said second soluble antigen or wherein the first of said two tagged polypeptides may comprise an antigen binding domain specific for an epitope C of said second soluble antigen and the second of said two tagged polypeptides may comprise an antigen binding domain specific for an epitope D of said second soluble antigen,

e) said second soluble antigen, wherein said second soluble antigen may comprise said epitope C and said epitope D,

wherein all tagged polypeptides may have the same tag,

or

wherein the composition of B may comprise additionally

d) a tagged polypeptide, wherein said tagged polypeptide may comprise an antigen binding domain specific for an epitope F of a second soluble antigen

e) said second soluble antigen, wherein said second soluble antigen may comprise at least two times said epitope F,

wherein all tagged polypeptides may have the same tag.

In one embodiment of the invention, the sole concentration of said first soluble antigen and the sole concentration of said second soluble antigen may be too low to activate the immune cell expressing said CAR that binds the sole soluble antigen, respectively. But the combination of both concentrations of the first soluble antigen and the second soluble antigen may be sufficient to activate the immune cell expressing said CAR by binding both soluble antigens via the same tag of the tagged polypeptides that bind to the soluble antigens, respectively.

Said compositions of A) and B) that may comprise said second soluble antigen as disclosed herein, wherein the concentration of the first soluble antigen is too low to solely activate said immune cell, and wherein the concentration of the second soluble antigen is too low to activate said immune cell, and wherein the sum of the concentrations of the first soluble antigen and the second soluble antigen is sufficient to activate that immune cell.

Said compositions may comprise further soluble antigens (e.g. a third, a fourth, a fifth, a sixth, a seventh or an eights soluble antigen) that may be bound by further tagged polypeptides having all the same tag, wherein merely the sum of concentrations of said further soluble antigens (e.g.

the sum of concentration of three, four, five, six, seven or eight soluble antigens) may activate said immune cell.

Said compositions of A) and B) that may comprise said second soluble antigen (or further soluble antigens) as disclosed herein, wherein said second soluble antigen (or a further soluble antigens) of said composition A and/or said second soluble antigen (or a further soluble antigens) of said composition B may be an antigen of a tumor microenvironment (TME) of a subject suffering from cancer, if the first soluble antigen may be an antigen of a tumor microenvironment (TME) of a subject suffering from cancer, or said second soluble antigen (or further soluble antigens) may be an antigen specifically associated with an autoimmune disease of a subject suffering from said autoimmune disease, if the first antigen may be an antigen specifically associated with an autoimmune disease of a subject suffering from said autoimmune disease, or said second soluble antigen (or further soluble antigens) may be an antigen specifically associated with an allergic disease of a subject suffering from said allergy, if the first antigen may be an antigen specifically associated with an allergic disease of a subject suffering from said allergy, or said second soluble antigen (or further soluble antigens) may be an antigen specifically associated with an infectious disease of a subject suffering from said infection, if the first antigen may be an antigen specifically associated with an infectious disease of a subject suffering from said infection, or said second soluble antigen (or further soluble antigens) may be an antigen specifically associated with a graft rejection in a subject suffering from said graft rejection, if the first antigen may be an antigen specifically associated with a graft rejection in a subject suffering from said graft rejection.

Said second soluble antigen (or further soluble antigens antigens) may be a cytokine, a chemokine, a shed surface receptor, extracellular matrix remodeling enzymes, a circulating tumor nucleic acid or tumor reactive antibody.

Said second soluble antigen (or further soluble antigens antigens) may be e.g. arginase, carcinoembryonic antigen, CCL11, CCL18, CCL2, CCL5, CD282, Circulating Tumor Nucleic Acids, CXCL10, FAP, GM-CSF, IFN-γ, IL-4, IL-6, IL-7 IL-8, IL-10, IL-11, IL-12, Il-13, IL-14, IL-15, IL-17, IL-23, IL-33, IL-1beta, IL-1Ra, INF, LAP, M-CSF, MMP12, MMP13, MMP7, NY-ESO-1 antibody, prostate-specific antigen, sCD106, sCD137, sCD152, sCD223, sCD25, sCD27, sCD253, sCD270, sCD273, sCD274, sCD279, sCD28, sCD30, sCD366, sCD40, sCD54, sCD80, sCD86, sGITR, TGFβ-1, TGFβ-2, TGFβ-3, TIMP1 or TNF-α, VEGF if said second soluble antigen (or further soluble antigens antigens) is a soluble antigen specifically associated with the TME.

Said second soluble antigen (or further soluble antigens antigens) may be e.g. CCL2, CCL3, CCL4, CCL5, CSCL11, CXCL10, CXCL8, CXCL9, GM-CSF, IL-1, IL10, IL-11, IL-12, IL-15, IL-17, IL-18, IL-2, IL-21, IL-22, IL-23, IL-6, IL-7, IL-8, INF or TNF, if said second soluble antigen (or further soluble antigens antigens) is a soluble antigen specifically associated with an autoimmune disease.

Said second soluble antigen (or further soluble antigens antigens) may be e.g. allergen specific IgE, eotaxin, GM-CSF, IFN, IL-13, IL-21, IL-31, IL-4, IL-5, IL-9, MCP-1, MCP-3, MCP-4, MDC, sCD23, sCD93 or TNF, if said second soluble antigen (or further soluble antigens antigens) is a soluble antigen specifically associated with an allergic disease.

Said second soluble antigen (or further soluble antigens antigens) may be e.g. CRP, IL-15, IL-27, IL-6, IL-7, IL-8, INF, sCD14, sCD163, soluble urokinase-type plasminogen activator receptor or sTREM-1, if said second soluble antigen (or further soluble antigens antigens) is a soluble antigen specifically associated with an infectious disease.

Said second soluble antigen (or further soluble antigens antigens) may be e.g. CCL2, CCL4, CXCL10, CXCL11, IL-2, IL-4, IL-6, IL-15, IL-18, IL-23, IFN, sCD4, sCD8, TNF or XCL1, if said second soluble antigen (or further soluble antigens antigens) is a soluble antigen specifically associated with graft rejection in a subject.

Said compositions of A) and/or B) as disclosed herein for use in the treatment of a cancer or an autoimmune disease or an allergic disease or an infectious disease or graft rejection in a subject.

An immune cell expressing said CAR as disclosed herein and activated by binding of the antigen binding domain of the CAR specific for a tag to a tagged polypeptide as disclosed herein that has attached thereto said soluble antigen as disclosed herein may secrete e.g. cytokines and chemokines that may exert effects on the environment said the activated immune cell (e.g. antiviral effects, anti-bacterial effects, trigger inflammation, stimulation of B cells, activation of macrophages, maturation of dendritic cells, healing of wounds, regulation of immune response, e.g. via regulatory T cells, induction of T cell proliferation).

In further embodiments of the invention and disclosed below said activated immune cell may be able to specifically express (and optionally secret) additional molecules as “effectors”.

Exemplary GPF has been used herein as an effector that has been induced after activation of the adCAR, but it is self-explaining that the effector may be any kind of nucleic acid and/or protein and/or peptide as disclosed herein.

Said compositions as disclosed herein, wherein said immune cell of the composition A) and/or the composition B) comprises polynucleotide(s) comprising:

a) said nucleic acid encoding said chimeric antigen receptor (CAR), wherein said nucleic acid comprises a constitutive promotor operably linked to the nucleic acid encoding said CAR, and

b) a second nucleic acid comprising an inducible promotor operably linked to a nucleic acid encoding an effector.

Said first nucleic acid and said second nucleic acid may be on one polynucleotide, e.g. on a single viral vector as disclosed e.g. in WO2019199689A1 or said first nucleic acid and said second nucleic acid may be on two separate polynucleotides, e.g. on two separate viral vectors.

Said compositions as disclosed herein, wherein said inducible promotor of the composition of A) may be capable of driving expression of the effector when the antigen binding domain of the CAR may bind to said tagged polypeptide(s) of A) that may be bound to said soluble antigen of A), or wherein said inducible promotor of the composition of B) may be capable of driving expression of the effector when the antigen binding domain of the CAR may bind to said tagged polypeptide of B) that may be bound to said soluble antigen of B).

Said compositions as disclosed herein, wherein the intracellular signaling domain of the CAR of the composition of A) may drive expression of the effector when the antigen binding domain of the CAR may bind to said tagged polypeptide(s) of A) that may be bound to said soluble antigen of A) by activating the inducible promotor operably linked to the effector, or wherein the intracellular signaling domain of the CAR of the composition of B) may drive expression of the effector when the antigen binding domain of the CAR may bind to said tagged polypeptide of B) that may be bound to said soluble antigen of B) by activating the inducible promotor operably linked to the effector.

Said compositions as disclosed herein, wherein said inducible promotor of composition A) may be capable of driving expression of the effector when the CAR of A) may bind said tagged polypeptide(s) of A) that may be bound to said soluble antigen of A), or wherein said inducible promotor of composition B) may be capable of driving expression of the effector when the CAR of B) may bind the said tagged polypeptide of B) that may be bound to said soluble antigen of B).

Said compositions as disclosed herein, wherein the inducible promoter of said composition A) and/or B) may comprise a promoter selected from the group consisting of an SP1, BATF, AP-1, IRF4, RUNX, NFAT, NF-κB, STAT5 or STAT3-sensing promotor and a minimal promoter.

Said compositions as disclosed herein, wherein the effector of composition A) and/or B) may be an antibody or antigen binding fragment thereof, a therapeutic peptide or protein, a cytokine, a chemokine, a receptor, a transcription factor, a siRNA, shRNA or extracellular matrix remodeling enzymes.

In a preferred embodiment of the invention the effector may be a receptor such as a CAR. Said CAR may be specific for an antigen expressed on a target cell. Said target cell may be a cancer cell.

Said composition as disclosed herein, wherein said immune cell of the composition A) and/or the composition B) comprises polynucleotide(s) comprising:

a) said nucleic acid encoding said chimeric antigen receptor (CAR), wherein said nucleic acid comprises a constitutive promoter operably linked to the nucleic acid encoding said CAR, and

b) a nucleic acid comprising an inducible promoter operably linked to a nucleic acid encoding a synthetic transcription factor for a drug-inducible promoter, wherein said synthetic transcription factor comprises a DNA binding domain and drug-binding domain and an activation domain, wherein said synthetic transcription factor is activated by binding to said drug, and

c) a nucleic acid comprising said drug-inducible promotor operably linked to a nucleic acid encoding an effector.

Said composition as disclosed herein, wherein said inducible promoter operably linked to a nucleic acid encoding a synthetic transcription factor of the composition of A) is capable of driving expression of the synthetic transcription factor when the antigen binding domain of the CAR binds to said tagged polypeptide(s) of A) that is/are bound to said soluble antigen of A), or wherein said inducible promoter operably linked to a nucleic acid encoding a synthetic transcription factor of the composition of B) is capable of driving expression of the synthetic transcription factor when the antigen binding domain of the CAR binds to said tagged polypeptide of B) that is bound to said soluble antigen of B).

Said composition as disclosed herein, wherein said inducible promoter operably linked to a nucleic acid encoding a synthetic transcription factor of said composition A) and/or B) may comprise a promoter selected from the group consisting of an SP1, BATF, AP-1, IRF4, RUNX, NFAT, NF-κB, STAT5 or STAT3-sensing promotor and a minimal promoter, and wherein said drug-inducible promoter may be a hybrid promoter comprising a DNA binding motif for said DNA binding domain and a minimal promoter (that comprises a TATA box, a initiator (Inr), TCT, BRE, MTE, or DPE motifs.)

Said DNA binding domain may be a protein or a portion of a protein that specifically recognize the DNA binding motif (DNA binding site) and mediate the binding of the synthetic transcription factor to this DNA sequence. Said DNA binding domain may be e.g. a zinc finger protein (or the DNA binding domain thereof) or a protein comprising or consisting of a POU domain.

Said composition as disclosed herein, wherein said synthetic transcription factor may comprise a zinc finger protein, the estrogen receptor (ER) and an activation domain, and wherein said drug may be tamoxifen or a tamoxifen metabolite.

Said activation domain may be e.g. herpes virus simplex protein VP16, the tetrameric repeat of VP16's minimal activation domain VP64, or the p65 domain of the human endogenous transcription factor NFκB.

Said tamoxifen metabolite may be endoxifen or 4-hydroxytamoxifen (4-OHT).

Said ER may be an ER having point mutations such as murine ER (G525R or G521R), human ER (G400V, M543A, L540A) or human ER (G400V, M543A, L544A).

Said drug-inducible promoter may be a hybrid promoter comprising a zinc finger binding motif and a minimal promoter that comprises a minimal promoter selected from the group consisting of E1b, TK, IL2, CMV, SV40.

Said composition as disclosed herein, wherein the effector of composition A) and/or B) may be an antibody or antigen binding fragment thereof, a therapeutic peptide or protein, a cytokine, a chemokine, a receptor, a transcription factor, a siRNA, or shRNA.

In another aspect, the invention provides a composition comprising

A)

a) an immune cell comprising polynucleotide(s) comprising:

I) a nucleic acid encoding a chimeric antigen receptor (CAR), wherein said nucleic acid comprises a constitutive promotor operably linked to the nucleic acid encoding said CAR, wherein said CAR comprises

i) an antigen binding domain specific for a tag of a tagged polypeptide or for two tagged polypeptides having the same tag

ii) a transmembrane domain

iii) an intracellular signaling domain,

II) a second nucleic acid comprising an inducible promotor operably linked to a nucleic acid encoding an effector, wherein said effector is expressed by said immune cell when said CAR is activated, and wherein said effector exerts an effect on a target cell

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen,

c) said soluble antigen, wherein said soluble antigen comprises said epitope A and said epitope B,

wherein said CAR is activated when said antigen binding domain of said CAR specific for the tag is bound to the tagged polypeptide(s) that has bound thereto said soluble antigen.

or

B)

a) an immune cell comprising polynucleotide(s) comprising:

I) a nucleic acid encoding a chimeric antigen receptor (CAR), wherein said nucleic acid comprises a constitutive promotor operably linked to the nucleic acid encoding said CAR, wherein said CAR comprises

i) an antigen binding domain specific for a tag of a tagged polypeptide

ii) a transmembrane domain

iii) an intracellular signaling domain

II) a second nucleic acid comprising an inducible promotor operably linked to a nucleic acid encoding an effector, wherein said effector is expressed by said immune cell when said CAR is activated, and wherein said effector exerts an effect on a target cell

b) said tagged polypeptide, wherein said tagged polypeptide comprises an antigen binding domain specific for an epitope E of a soluble antigen

c) said soluble antigen, wherein said soluble antigen comprises at least two times said epitope E,

wherein said CAR is activated when said antigen binding domain of said CAR specific for the tag is bound to the tagged polypeptide that has bound thereto said soluble antigen.

Said target cell may be said immune cell itself, a tumor cell, a cell associated with an autoimmune disease, a cell associated with an allergic disease, a cell associated with an infectious disease (e.g. an infected cell), a cell associated with graft rejection in a subject.

Said effect on said target cell may be a stimulation and/or proliferation of the target cell when the target cell is said immune cell itself.

Said effect on said target cell may be an increased cytokine production and/or chemokine production and/or killing activity of the immune cell towards the target cell, when the target cell is tumor cell and/or a TME related cell.

Said effect on said target cell may be an increased cytokine production and/or chemokine production and/or stimulation of anti-inflammatory cells e.g. regulatory T cells when the target cell is a cell associated with an autoimmune disease.

Said effect on said target cell may be increased cytokine production and/or chemokine production and/or stimulation of immune cells reactive towards the e.g. the infected cell, when the target cell is a cell associated with an infectious disease (e.g. an infected cell).

Said effect on said target cell may be an increased cytokine production and/or chemokine production and/or stimulation of anti-inflammatory cells e.g. regulatory T cells and/or inhibiting cells reactive towards an allergen e.g. mast cells, when the target cell is a cell associated with an allergic disease.

Said effect on said target cell may be an increased cytokine production and/or chemokine production and/or stimulation of anti-inflammatory cells e.g. regulatory T cells, when the target cell is a cell associated with graft rejection in a subject.

Said soluble antigen may be a molecule secreted by said target cell.

In a further aspect the present invention provides a composition comprising

A)

a) an immune cell expressing a chimeric antigen receptor (CAR) comprising

i) an antigen binding domain specific for a tag of a tagged polypeptide or for two tagged polypeptides having the same tag

ii) a transmembrane domain

iii) an intracellular signaling domain

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen,

c) said soluble antigen, wherein said soluble antigen comprises said epitope A and said epitope B,

or

B)

a) an immune cell expressing a chimeric antigen receptor (CAR) comprising

i) an antigen binding domain specific for a tag of a tagged polypeptide

ii) a transmembrane domain

iii) an intracellular signaling domain

b) said tagged polypeptide, wherein said tagged polypeptide comprises an antigen binding domain specific for an epitope E of a soluble antigen

c) said soluble antigen, wherein said soluble antigen comprises at least two times said epitope E.

In a further aspect the present invention provides a composition for use in the treatment of a cancer or an autoimmune disease or an allergic disease or an infectious disease or a graft rejection in a subject comprising

A)

a) an immune cell expressing a chimeric antigen receptor (CAR) comprising

i) an antigen binding domain specific for a tag of a tagged polypeptide or for two tagged polypeptides having the same tag

ii) a transmembrane domain

iii) an intracellular signaling domain

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen,

c) said soluble antigen, wherein said soluble antigen comprises said epitope A and said epitope B,

or

B)

a) an immune cell expressing a chimeric antigen receptor (CAR) comprising

i) an antigen binding domain specific for a tag of a tagged polypeptide

ii) a transmembrane domain

iii) an intracellular signaling domain

b) said tagged polypeptide, wherein said tagged polypeptide comprises an antigen binding domain specific for an epitope E of a soluble antigen

c) said soluble antigen, wherein said soluble antigen comprises at least two times said epitope E.

In another aspect the present invention provides a method for treating a subject suffering from cancer or an autoimmune disease or an allergic disease or an infectious disease or a graft rejection in a subject, the method comprising

A) administering to said subject

a) an immune cell comprising a polynucleotide comprising a nucleic acid encoding a chimeric antigen receptor (CAR) comprising

i) an antigen binding domain specific for a tag of a tagged polypeptide or for two tagged polypeptides having the same tag

ii) a transmembrane domain

iii) an intracellular signaling domain, and

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen, wherein said soluble antigen comprises said epitope A and said epitope B,

or

B) administering to said subject

a) an immune cell comprising a polynucleotide comprising a nucleic acid encoding a chimeric antigen receptor (CAR) comprising

i) an antigen binding domain specific for a tag of a tagged polypeptide

ii) a transmembrane domain

iii) an intracellular signaling domain, and

b) said tagged polypeptide, wherein said tagged polypeptide comprises an antigen binding domain specific for an epitope E of a soluble antigen, wherein said soluble antigen comprises at least two times said epitope E.

Said immune cell may also comprise the nucleic acid comprising said drug-inducible promotor operably linked to a nucleic acid encoding an effector as disclosed herein.

Said administration of the immune cell of A) and said administration of said tagged polypeptide(s) of A) to the subject may be performed simultaneously or subsequently. If administered subsequently the immune cell may be administered before the tagged polypeptide or the immune cell may be administered after the tagged polypeptide

Said administration of the immune cell of B) and said administration of said tagged polypeptide of B) to the subject may be performed simultaneously or subsequently. If administered subsequently the immune cell may be administered before the tagged polypeptide or the immune cell may be administered after the tagged polypeptide

In another aspect the present invention provides a method for treating a subject suffering from cancer or an autoimmune disease or an allergic disease or an infectious disease or a graft rejection, the method comprising

A) administering to said subject

a) an immune cell expressing a chimeric antigen receptor (CAR) comprising

i) an antigen binding domain specific for a tag of a tagged polypeptide or for two tagged polypeptides having the same tag

ii) a transmembrane domain

iii) an intracellular signaling domain, and

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen, wherein said soluble antigen comprises said epitope A and said epitope B,

or

B) administering to said subject

a) an immune cell expressing a chimeric antigen receptor (CAR) comprising

i) an antigen binding domain specific for a tag of a tagged polypeptide

ii) a transmembrane domain

iii) an intracellular signaling domain, and

b) said tagged polypeptide, wherein said tagged polypeptide comprises an antigen binding domain specific for an epitope E of a soluble antigen, wherein said soluble antigen comprises at least two times said epitope E.

Said administration of the immune cell of A) and said administration of said tagged polypeptide(s) of A) to the subject may be performed simultaneously or subsequently. If administered subsequently the immune cell may be administered before the tagged polypeptide or the immune cell may be administered after the tagged polypeptide

Said administration of the immune cell of B) and said administration of said tagged polypeptide of B) to the subject may be performed simultaneously or subsequently. If administered subsequently the immune cell may be administered before the tagged polypeptide or the immune cell may be administered after the tagged polypeptide

In another aspect the present invention provides a pharmaceutical composition comprising

A)

a) an immune cell comprising a polynucleotide comprising a nucleic acid encoding a chimeric antigen receptor (CAR) comprising

i) an antigen binding domain specific for a tag of a tagged polypeptide or for two tagged polypeptides having the same tag

ii) a transmembrane domain

iii) an intracellular signaling domain

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen, wherein said soluble antigen comprises said epitope A and said epitope B,

or

B)

a) an immune cell comprising a polynucleotide comprising a nucleic acid encoding a chimeric antigen receptor (CAR) comprising

i) an antigen binding domain specific for a tag of a tagged polypeptide

ii) a transmembrane domain

iii) an intracellular signaling domain

b) said tagged polypeptide, wherein said tagged polypeptide comprises an antigen binding domain specific for an epitope E of a soluble antigen, wherein said soluble antigen comprises at least two times said epitope E.

Said compositions may optionally comprise a pharmaceutical acceptable carrier.

In another aspect the present invention provides a pharmaceutical composition comprising

A)

a) an immune cell expressing a chimeric antigen receptor (CAR) comprising

i) an antigen binding domain specific for a tag of a tagged polypeptide or for two tagged polypeptides having the same tag

ii) a transmembrane domain

iii) an intracellular signaling domain

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen, wherein said soluble antigen comprises said epitope A and said epitope B,

or

B)

a) an immune cell expressing a chimeric antigen receptor (CAR) comprising

i) an antigen binding domain specific for a tag of a tagged polypeptide

ii) a transmembrane domain

iii) an intracellular signaling domain

b) said tagged polypeptide, wherein said tagged polypeptide comprises an antigen binding domain specific for an epitope E of a soluble antigen, wherein said soluble antigen comprises at least two times said epitope E.

Said compositions may optionally comprise a pharmaceutical acceptable carrier.

In another aspect, the invention provides a kit comprising

A)

a) an immune cell comprising a polynucleotide comprising a nucleic acid encoding a chimeric antigen receptor (CAR) comprising

i) an antigen binding domain specific for a tag of a tagged polypeptide or for two tagged polypeptides having the same tag

ii) a transmembrane domain

iii) an intracellular signaling domain

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen, wherein said soluble antigen comprises said epitope A and said epitope B,

or

B)

a) an immune cell comprising a polynucleotide comprising a nucleic acid encoding a chimeric antigen receptor (CAR) comprising

i) an antigen binding domain specific for a tag of a tagged polypeptide

ii) a transmembrane domain

iii) an intracellular signaling domain

b) said tagged polypeptide, wherein said tagged polypeptide comprises an antigen binding domain specific for an epitope E of a soluble antigen, wherein said soluble antigen comprises at least two times said epitope E.

In another aspect, the invention provides a kit comprising

A)

a) a polynucleotide comprising a nucleic acid encoding a chimeric antigen receptor (CAR) comprising

i) an antigen binding domain specific for a tag of a tagged polypeptide or for two tagged polypeptides having the same tag

ii) a transmembrane domain

iii) an intracellular signaling domain

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen, wherein said soluble antigen comprises said epitope A and said epitope B,

or

B)

a) a polynucleotide comprising a nucleic acid encoding a chimeric antigen receptor (CAR) comprising

i) an antigen binding domain specific for a tag of a tagged polypeptide

ii) a transmembrane domain

iii) an intracellular signaling domain

b) said tagged polypeptide, wherein said tagged polypeptide comprises an antigen binding domain specific for an epitope E of a soluble antigen, wherein said soluble antigen comprises at least two times said epitope E.

In another aspect, the invention provides a kit comprising

A)

a) an immune cell expressing a chimeric antigen receptor (CAR) comprising

i) an antigen binding domain specific for a tag of a tagged polypeptide or for two tagged polypeptides having the same tag

ii) a transmembrane domain

iii) an intracellular signaling domain

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen, wherein said soluble antigen comprises said epitope A and said epitope B,

or

B)

a) an immune cell expressing a chimeric antigen receptor (CAR) comprising

i) an antigen binding domain specific for a tag of a tagged polypeptide

ii) a transmembrane domain

iii) an intracellular signaling domain

b) said tagged polypeptide, wherein said tagged polypeptide comprises an antigen binding domain specific for an epitope E of a soluble antigen, wherein said soluble antigen comprises at least two times said epitope E.

In one embodiment of the invention, the compositions as disclosed herein comprising immune cells comprising the CAR specific for a tag of a tagged polypeptide (“anti-tag CAR”) as disclosed herein and said tagged polypeptide(s) specifically binding to the epitope(s) of a soluble antigen as disclosed herein may be for use e.g. in the treatment of a subject suffering from cancer or an autoimmune disease or an allergic disease or an infectious disease or a graft rejection as disclosed herein. Cells such as immune cells, e.g. T cells, NK cells or Treg cells of a subject, may be isolated or established immune cell lines may be used. The subject may suffer from said cancer (a patient) or said autoimmune disease or may be a healthy subject. These immune cells are genetically modified in vitro to express said CAR as disclosed herein. These engineered cells may be activated and expanded in vitro to a therapeutically effective population of expressing cells. In cellular therapy these engineered cells may be infused to a recipient in need thereof as a pharmaceutical composition (or a formulation of a therapeutically effective population of the CAR expressing cells), in addition to a second pharmaceutical composition, a formulation of the tagged polypeptide(s) as disclosed herein. The infused cells in the recipient may be e.g. able to kill (or at least stop growth of) cancerous cells expressing the antigen which is recognized by the CAR system as disclosed herein or may reduce the effect of the autoimmune disease. The recipient may be the same subject from which the cells were obtained (autologous cell therapy) or may be from another subject of the same species (allogeneic cell therapy).

The therapeutically effective population of CAR expressing cells may be administered to the patient before the administration of the formulation of the tagged polypeptide(s) to the subject. Alternatively, the formulation of the tagged polypeptide(s) may be administered to the subject before or at the same time as the administration the therapeutically effective population of said CAR expressing cells to the subject. A further variation includes in-vitro culturing the therapeutically effective population of said CAR expressing cells with the tagged polypeptide(s) of the formulation of the tagged polypeptide(s) prior to administration to the subject.

Populations of said CAR expressing (immune) cells may be formulated for administration to a subject using techniques known to the skilled artisan.

Formulations comprising therapeutically effective population(s) of said CAR expressing cells may include pharmaceutically acceptable excipient(s) (carrier or diluents). Excipients included in the formulations will have different purposes depending, for example, on the nature of the tag-binding domain of the anti-tag-CAR, the (sub)population of immune cells used, and the mode of administration. Examples of generally used excipients include, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents.

A formulation of a therapeutically effective population(s) of said CAR expressing cells may include one population of said CAR expressing (immune) cells, or more than one population of said CAR expressing (immune) cells. The different populations of said CAR expressing (immune) cells may vary based on the identity of the activation domain, the identity of the (sub)population of immune cells, or a combination thereof.

The formulations comprising therapeutically effective population(s) of said CAR expressing cells may be administered to a subject using modes and techniques known to the skilled artisan. Exemplary modes include, but are not limited to, intravenous injection. Other modes include, without limitation, intratumoral, intradermal, subcutaneous (s.c, s .q., sub-Q, Hypo), intramuscular (i.m.), intraperitoneal (i.p.), intra-arterial, intramedulary, intracardiac, intra-articular (joint), intrasynovial (joint fluid area), intracranial, intraspinal, and intrathecal (spinal fluids).

The formulations comprising therapeutically effective population(s) of said CAR expressing cells that are administered to a subject comprise a number of said CAR expressing cells such immune cells that is effective for the treatment of the specific indication or disorder.

In general, formulations may be administered that comprise between about 1×10⁴ and about 1×10¹⁰ said CAR expressing cells such as immune cells. In most cases, the formulation may comprise between about 1×10⁵ and about 1×10⁹ said CAR expressing cells such as immune cells, from about 5×10⁵ to about 5×10⁸ said CAR expressing cells such as immune cells, or from about 1×10⁶ to about 1×10⁷ said CAR expressing cells such as immune cells. However, the number of said CAR expressing cells such as immune cells administered to a subject may vary between wide limits, depending upon the location, source, identity, extent and severity of the disorder, the age and condition of the individual to be treated, etc. A physician may ultimately determine appropriate dosages to be used.

The tagged polypeptide(s) as disclosed herein may be formulated for administered to a subject using techniques known to the skilled artisan. Formulations of the tagged polypeptide(s) may include pharmaceutically acceptable excipient(s) (carriers or diluents). Excipients included in the formulations will have different purposes depending, for example, on the nature of the tag, the antigen binding domain of the tagged polypeptide, and the mode of administration. Examples of generally used excipients include, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents.

A formulation of tagged polypeptide may include one type of tag polypeptide, or more than one type of tagged polypeptides. The different types of tagged polypeptides may vary based on the identity of the antigen binding moiety of the tagged polypeptide.

The tagged polypeptide(s) may be administered to a subject using modes and techniques known to the skilled artisan. Exemplary modes include, but are not limited to, intravenous, intraperitoneal, and intratumoral injection. Other modes include, without limitation, intradermal, subcutaneous (s.c, s.q., sub-Q, Hypo), intramuscular (i.m.), intra-arterial, intramedulary, intracardiac, intra-articular (joint), intrasynovial (joint fluid area), intracranial, intraspinal, and intrathecal (spinal fluids).

Formulations comprising the polypeptide(s) are administered to a subject in an amount which is effective for treating the specific indication or disorder. In general, formulations comprising at least about 1 μg/kg to about 100 mg/kg body weight of the tagged polypeptide(s) may be administered to a subject in need of treatment. In most cases, the dosage may be from about 100 μg/kg to about 10 mg/kg body weight of the tagged polypeptide(s) daily, taking into account the routes of administration, symptoms, etc. The amount of tagged polypeptide(s) in formulations administered to a subject may vary between wide limits, depending upon the location, source, identity, extent and severity of the disorder, the age and condition of the individual to be treated, etc. A physician may ultimately determine appropriate dosages to be used.

The timing between the administration of the CARs expressing cell formulation and the tag polypeptide-formulation may range widely depending on factors that include the type of (immune) cells being used, the binding specificity of the CARs, the identity of the tag, the antigen binding moiety of the tagged polypeptide, the identity of said soluble antigen, the identity of the target cell, e.g. cancer cell to be treated, the location of the target cell in the subject, the means used to administer the formulations to the subject, and the health, age and weight of the subject being treated. Indeed, the tagged polypeptide formulation may be administered prior to, simultaneous with, or after the genetically engineered (immune) cell formulation.

Depending on the disorder being treated the step of administering the CARs expressing cell formulation, or the step of administering the tagged polypeptide formulation, or both, can be repeated one or more times. When two or more formulations of engineered cells such as immune cells expressing the CARs of the invention are applied to a subject, the engineered cells may be of the same cell type or of different cell types, e.g. T cells and/or NK cells. A formulation of cells such as immune cells may also comprise more than one cell type, each expressing the CARs of the invention.

In a further aspect the present invention provides a composition comprising

A)

a) an immune cell comprising a polynucleotide comprising a nucleic acid encoding a chimeric antigen receptor (CAR) comprising

i) an antigen binding domain specific for a tag of a tagged polypeptide or for two tagged polypeptides having the same tag

ii) a transmembrane domain

iii) an intracellular signaling domain

b) said tagged polypeptide or said two tagged polypeptides, wherein said tagged polypeptide comprises an antigen binding domain specific for an epitope A of a soluble antigen and an antigen binding domain specific for an epitope B of said soluble antigen or wherein the first of said two tagged polypeptides comprises an antigen binding domain specific for an epitope A of said soluble antigen and the second of said two tagged polypeptides comprises an antigen binding domain specific for an epitope B of said soluble antigen,

c) said soluble antigen, wherein said soluble antigen comprises said epitope A and said epitope B,

or

B)

a) an immune cell comprising a polynucleotide comprising a nucleic acid encoding a chimeric antigen receptor (CAR) comprising

i) an antigen binding domain specific for a tag of a tagged polypeptide

ii) a transmembrane domain

iii) an intracellular signaling domain

b) said tagged polypeptide, wherein said tagged polypeptide comprises an antigen binding domain specific for an epitope E of a soluble antigen

c) said soluble antigen, wherein said soluble antigen comprises at least two times said epitope E;

wherein the intracellular signaling domain of said CAR of A) and/or B) comprises at least one inhibitory endodomain, wherein said at least one inhibitory endodomain is a cytoplasmic signaling domain comprising at least one signal transduction element that inhibits an immune cell or comprising at least one element that induces apoptosis,

and wherein said immune cell of A) and/or B) comprise a polynucleotide comprising a nucleic acid encoding a second CAR comprising

i) an antigen binding domain specific for an antigen expressed on a target cell

ii) a transmembrane domain

ii) an intracellular signaling domain comprising at least one primary cytoplasmic signaling domain comprising an immunoreceptor tyrosine-based activation motif (ITAM).

Said second CAR may be an activating CAR (a standard CAR) as described above. Said CAR specific for said tag and said second CAR may be constitutively expressed in said immune cell.

Said target cell may be a cancer cell or a cell associated with an autoimmune disease or an allergic disease or an infectious disease or a graft rejection in a subject.

Said soluble antigen of composition A) and/or B) may be soluble antigen secreted by cells in an environment of a subject that is not a TME of said subject (non-target cells). Therefore, the immune cells expressing said adapterCAR and said second CAR specific for an antigen on a target cell are not directed against cells of said environment that is not a TME.

Inhibitory endodomains of an iCAR are well-known in the art and have been described e.g. in WO2015075469A1, WO2015075470A1, WO2015142314A1, WO2016055551A1, WO2016097231A1, WO2016193696A1, WO2017058753A1, WO2017068361A1, WO2018061012A1, and WO2019162695A1.

Said at least one signal transduction element that inhibits or may be capable of inhibiting an (effector) immune cell of said iCAR may be a signal transduction element of an immune checkpoint protein.

Said inhibitory signal transduction element may be selected from the groups consisting of:
the immunoglobulin superfamily (IgSF) and tumour necrosis factor receptor superfamily (TNFRSF) including immune checkpoint proteins CD22, CD31, CD33, CD47, CD85A (LIR3), CD85C (LIR8), CD85D (LIR2), CD87J (LIR1), CD85K (LIR5), CD89 (B71), CD94 (KLRD1), CD152 (CTLA4), CD158A (KIR2DL1), CD158B1 (KIR2DL2), CD158B2 (KIR2DL3), CD158D (KIR2DL4), CD158E1 (KIR3DL1), CD158F (KIR2DL5A) CD158K (KIR3DL2), CD158Z (KIR3DL3), CD159a, CD159c, CD160, CD223 (LAG3), CD244 (SLAMF4), CD272 (BTLA), CD274 (PDL1), CD279 (PD1), CD328 (Siglec7), CD329 (Siglec9), CD352 (SLAMF6), CEACAM1, CEACAM2, FcgammaR, G6b-B, KIR2DL5B, KLRG1, LAIR1, PD1H (Vista), PIR-B, Siglec2, Siglec3, Siglec5, Siglec6, Siglec8, Siglec10, Siglec11, Siglec12, TIGIT, TIM2, TIM3, and TLT-1
protein tyrosine phosphatases ACP1, CDC14A, CDC14B, CDC14C, CDC25A, CDC25B, CDC25C, CDKN3, DNAJC6, DUPD1, DUSP1, DUSP10, DUSP11, DUSP12, DUSP13, DUSP14, DUSP15, DUSP16, DUSP18, DUSP19, DUSP2, DUSP21, DUSP22, DUSP23, DUSP26, DUSP27, DUSP28, DUSP3, DUSP4, DUSP5, DUSP6, DUSP7, DUSP8, DUSP9, EPM2A, FIG4, GAK, INPP5A, INPP5B, INPP5D, INPP5E, INPP5F, INPP5J, INPP5K, INPPL1, MTM1, MTMR1, MTMR10, MTMR11, MTMR12, MTMR14, MTMR2, MTMR3, MTMR4, MTMR6, MTMR7, MTMR8, MTMR9, OCRL, PALD1, PIP4P1, PIP4P2, PTEN, PTP4A1, PTP4A2, PTP4A3, PTPDC1, PTPMT1, PTPN1, PTPN11, PTPN12, PTPN13, PTPN14, PTPN18, PTPN2, PTPN20, PTPN21, PTPN22, PTPN23, PTPN3, PTPN4, PTPN5, PTPN6, PTPN7, PTPN9, PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRZ1, RNGTT, SACM1L, SBF1, SBF2, SSH1, SSH2, SSH3, STYX, STYXL1, SYNJ1, SYNJ2, TNS1, TNS2, TNS3, TNS4, TPTE, and TPTE2.

Said at least one signal transduction element that inhibits an immune cell of said iCAR may be also selected from STimulator of INterteron Genes (STING); immunoreceptor tyrosine-based inhibitory motif (ITIM) containing proteins, immunoreceptor tyrosine-based switch motif (ITSM) containing proteins, T cell immunoglobulin and IITM domain (TIGIT), and adenosine receptor (e.g. A2aR).

Said at least one signal transduction element that inhibits an immune cell of said iCAR may be also a tyrosine phosphatase domain from a Src homolog (SH2) domain-containing protein tyrosine phosphatase which is recruited by a phosphorylated Immunoreceptor Tyrosine-based Activation motif (ITIM).

Said at least one signal transduction element that inhibits an immune cell of said iCAR may be also

(i) a truncated protein which comprises an SH2 domain from a protein which binds a phosphorylated immunoreceptor tyrosine-based activation motif (ITAM), but lacks a kinase domain; or

(ii) a truncated protein which comprises an SH2 domain from a protein which binds a phosphorylated immunoreceptor tyrosine-based inhibition motif (ITIM) but lacks a phosphatase domain; or

(iii) a fusion protein which comprises (a) an SH2 domain from a protein which binds a phosphorylated immunoreceptor tyrosine-based activation motif (ITAM) or from a protein which binds a phosphorylated immunoreceptor tyrosine-based inhibition motif (ITIM); and (ii) a heterologous domain. Said heterologous domain may be a phosphatase domain or a kinase domain.

Said at least one element that induces apoptosis may be e.g. a Tumor-necrosis-factor related apoptosis inducing ligand (TRAIL) receptor or a CD200 receptor as described e.g. in detail in WO20160972331A1.

All definitions, characteristics and embodiments defined herein with regard to the first aspect of the invention as disclosed herein also apply mutatis mutandis in the context of the other aspects of the invention as disclosed herein.

In addition to above described applications and embodiments of the invention further embodiments of the invention are described in the following without intention to be limited to these embodiments.

### Embodiments

In one embodiment of the invention, the immune cell, e.g. a T cell comprises a first nucleic encoding an anti-tagCAR, e.g. an anti-biotin-CAR, a second nucleic acid comprising an inducible promotor operably linked to a nucleic acid encoding a second CAR as effector. Said second CAR is specific for a tumor-associated antigen (TAA) expressed on the surface of a cancer cell that is part of a solid tumor. The soluble LAP is present in high concentrations in the tumor-microenvironment of a subject. In presence of an adapter that is composed of a biotinylated antibody or antigen binding fragment thereof specific for said soluble antigen the anti-tag CAR is activated in the TME upon binding of the adapter that has bound to the soluble antigen and the expression of the second CAR is induced in the immune cell. After expression of the second CAR that is specific for the TAA of the solid tumor, the immune cell exerts cytotoxic activity against the solid tumor.

In one embodiment of the invention, the immune cell, e.g. a T cell comprises a first nucleic encoding an anti-tagCAR, e.g. an anti-biotin-CAR, a second nucleic acid comprising an inducible promotor operably linked to a nucleic acid encoding a second CAR as effector. Said second CAR is specific for a tumor-associated antigen (TAA) expressed on the surface of a cancer cell that is part of a solid tumor. The soluble LAP can temporally be immobilized e.g. on integrins. In presence of an adapter that e.g. is composed of a biotinylated antibody or antigen binding fragment thereof specific for said soluble antigen the anti-tag CAR is activated in the TME upon binding of the adapter that has bound to the soluble antigen and the expression of the second CAR is induced in the immune cell. The activation of the anti-tag CAR is independent of the immobilized LAP and does not require the immobilization. After expression of the second CAR that is specific for the TAA of the solid tumor, the immune cell exerts cytotoxic activity against the solid tumor.

In one embodiment of the invention, the immune cell, e.g. a T cell comprises a first nucleic encoding an anti-tag CAR, e.g. an anti-biotin-CAR, a second nucleic acid comprising an inducible promotor operably linked to a nucleic acid encoding for a cytokine and/or a chemokine. Said cytokine or chemokine is specifically expressed in the TME. The soluble LAP is present in high concentrations in the tumor-microenvironment of a subject. In presence of an adapter that is composed of a biotinylated antibody or antigen binding fragment thereof specific for said soluble antigen the anti-tag CAR is activated in the TME upon binding of the adapter that has bound to the soluble antigen and the expression of the cytokine and/or chemokine is induced in the immune cell. After expression of the cytokine and/or chemokine a local controlled inflammation is induced in the TME. The altered TME enhances subsequently the penetration of the tumor and/or the cytotoxicity of immune cells e.g. CAR T cells or innate immune cells.

In one embodiment of the invention, the immune cell, e.g. a T cell comprises a first nucleic encoding an anti-tagCAR, e.g. an anti-biotin-CAR, a second nucleic acid comprising an inducible promotor operably linked to a nucleic acid encoding a synthetic transcription factor that may comprise a zinc finger protein, the estrogen receptor (ER), an activation domain, and a drug inducible promotor with linked effector molecule. Said inducible synthetic transcription factor can bind to its dedicated DNA binding site and induce gene expression downstream of the drug inducible promotor.

The soluble LAP is present in high concentrations in the tumor-microenvironment of a subject. In presence of an adapter that is composed of a biotinylated antibody or antigen binding fragment thereof specific for said soluble antigen the anti-tag CAR is activated in the TME upon binding of the adapter that has bound to the soluble antigen and the expression of said inducible synthetic transcription factor that may comprise a zinc finger protein, the estrogen receptor (ER) and an activation domain is induced in the immune cell. By adding the inducer drug, local, dose and time-controlled transcription of a transgene is induced wherein said drug may be tamoxifen or a tamoxifen metabolite.

In one embodiment of the invention, the immune cell, e.g. a T cell comprises a first nucleic encoding an anti-tagCAR, e.g. an anti-biotin-CAR, a second nucleic acid comprising an inducible promotor operably linked to a nucleic acid encoding for an anti-inflammatory molecule e.g. a chemokine, cytokine or a soluble receptor. The soluble antigen e.g. CXCL10 is present in high concentrations at the site of inflammation. In presence of an adapter that is composed of a biotinylated antibody or antigen binding fragment thereof specific for said soluble antigen the anti-tag CAR is activated at the site of inflammation upon binding of the adapter that has bound to the soluble antigen the expression of anti-inflammatory e.g. TGF-β, is induced. The local immunosuppression can be used to treat e.g. autoimmune diseases.

In one embodiment of the invention, the immune cell, e.g. a regulatory T cell comprises a first nucleic encoding an anti-tagCAR, e.g. an anti-biotin-CAR, a second nucleic acid comprising an inducible promotor operably linked to a nucleic acid encoding for an anti-inflammatory molecule e.g. a chemokine, cytokine or a soluble receptor. The soluble antigen e.g. CXCL9 is present in high concentrations at the site of the graft. In presence of an adapter that is composed of a biotinylated antibody or antigen binding fragment thereof specific for said soluble antigen the anti-tag CAR is activated at the site of the graft upon binding of the adapter that has bound to the soluble antigen the expression of anti-inflammatory e.g. TGF-β, is induced. The local immunosuppression can be used to treat e.g. a graft rejection.

Definitions

Unless defined otherwise, technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not.

In general, a CAR may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In the embodiments of the present invention the antigen binding domain of the CAR binds a tag or hapten that is coupled to a polypeptide (“haptenylated” or “tagged” polypeptide), wherein the polypeptide may bind to an epitope of a soluble antigen as disclosed herein.

The CAR as disclosed herein may be referred to as “anti-tag” CAR or “adapterCAR” or “universal CAR” as disclosed e.g. in U.S. Pat. No. 9,233,125B2.

The haptens or tags may be coupled directly or indirectly to a polypeptide (the tagged polypeptide), wherein the polypeptide may bind to said epitope of a soluble antigen. The tag may be e.g. dextran or a hapten such as biotin or fluorescein isothiocyanate (FITC) or phycoerythrin (PE), but the tag may also be a peptide sequence e.g. chemically or recombinantly coupled to the polypeptide part of the tagged polypeptide. The tag may also be streptavidin. The tag portion of the tagged polypeptide is only constrained by being a molecule that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv, respectively.

A “signal peptide” refers to a peptide sequence that directs the transport and localization of the protein within a cell, e.g. to a certain cell organelle (such as the endoplasmic reticulum) and/or the cell surface.

Generally, an “antigen binding domain” refers to the region of the CAR that specifically binds to an antigen, e.g. to a soluble antigen as disclosed herein. Generally, the targeting regions on the CAR are extracellular. The antigen binding domain may comprise an antibody or an antigen binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S)₃-linker”.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

“Spacer” or “hinge” as used herein refers to the hydrophilic region which is between the antigen binding domain and the transmembrane domain. The CARs of the invention may comprise an extracellular spacer domain but it is also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. The splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (the intracellular signaling domain or the activating endodomain) of the CAR is responsible for activation of at least one of the normal effector functions of the immune cell in which the CAR is expressed, if the respective CAR is an activating CAR (normally, a CAR as described herein refers to an activating CAR). “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRζ (CD3ζ), FcRgamma, FcRbeta, CD3gamma, CD3delta, CD3epsilon, CD5, CD22, CD79a, CD79b, and CD66d. Most prominent is sequence derived from CD3ζ (CD3zeta).

The cytoplasmic domain of the CAR may be designed to comprise the CD3ζ signaling domain by itself or combined with any other desired cytoplasmic domain(s). The cytoplasmic domain of the CAR can comprise a CD3ζ chain portion and a co-stimulatory signaling region (domain). The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

As aforementioned either the extracellular part or the transmembrane domain or the cytoplasmic domain of a CAR may also comprise a heterodimerizing domain for the aim of splitting key signaling and antigen recognition modules of the CAR.

The CAR may be further modified to include on the level of the nucleic acid encoding the CAR one or more operative elements to eliminate CAR expressing immune cells by virtue of a suicide switch. The suicide switch can include, for example, an apoptosis inducing signaling cascade or a drug that induces cell death. In one embodiment, the nucleic acid expressing and encoding the CAR can be further modified to express an enzyme such thymidine kinase (TK) or cytosine deaminase (CD). The CAR may also be part of a gene expression system that allows controlled expression of the CAR in the immune cell. Such a gene expression system may be an inducible gene expression system and wherein when an induction agent is administered to a cell being transduced with said inducible gene expression system, the gene expression system is induced and said CAR is expressed on the surface of said transduced cell.

In some embodiments, the endodomain may contain a primary cytoplasmic signaling domains or a co-stimulatory region, but not both. In these embodiments, an immune effector cell containing the disclosed CAR is only activated if another CAR containing the missing domain also binds its respective antigen.

In some embodiment of the invention the CAR may be a “SUPRA” (split, universal, and programmable) CAR, where a “zipCAR” domain may link an intra-cellular costimulatory domain and an extracellular leucine zipper (WO2017/091546). This zipper may be targeted with a complementary zipper fused e.g. to an scFv region to render the SUPRA CAR T cell specific for the soluble antigen.

If the immune cell expresses a second, induced CAR as disclosed herein, then the second CAR may comprise an antigen binding domain that specifically binds to an antigen, e.g. an antigen expressed on the surface of a target cell, e.g. a tumor cell.

In some embodiments the CAR may also be an inhibitory CAR (iCAR) as disclosed herein.

The CARs of the present invention may be designed to comprise any portion or part of the above-mentioned domains as described herein in any order and/or combination resulting in a functional CAR, i.e. a CAR that mediated an immune effector response of the immune effector cell that expresses the CAR as disclosed herein or that has an inhibitory function (iCAR) as disclosed herein.

The term “tagged polypeptide” as used herein refers to a polypeptide that has bound thereto directly or indirectly at least one additional component, i.e. the tag. The tagged polypeptide as used herein is able to bind an epitope of a soluble antigen as disclosed herein. The polypeptide may be an antibody or antigen binding fragment thereof that binds to said epitope of the soluble antigen. The polypeptide of the tagged polypeptide alternatively may a ligand binding receptor, that can bind the soluble ligand, e.g. a cytokine receptor that binds the corresponding soluble cytokine.

The terms “adapter” or “adapter molecule” or “tagged polypeptide” as used herein may be used interchangeably.

The tag may be e.g. a hapten or dextran and the hapten or dextran may be bound by the antigen binding domain of the polypeptide, e.g. a CAR, comprising an antigen binding domain specific for the tag.

Haptens such as e.g. FITC, biotin, PE, streptavidin or dextran are small molecules that elicit an immune response only when attached to a large carrier such as a protein; the carrier may be one that also does not elicit an immune response by itself. Once the body has generated antibodies to a hapten-carrier adduct, the small-molecule hapten may also be able to bind to the antibody, but it will usually not initiate an immune response; usually only the hapten-carrier adduct can do this.

But the tag may also be a peptide sequence e.g. chemically or recombinantly coupled to the polypeptide part of the tagged polypeptide. The peptide may be selected from the group consisting of c-Myc-tag, Strep-Tag, Flag-Tag, and Polyhistidine-tag. The tag may also be streptavidin. The tag portion of the tagged polypeptide is only constrained by being a molecule that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv.

The term “antibody” as used herein is used in the broadest sense to cover the various forms of antibody structures including but not being limited to monoclonal and polyclonal antibodies (including full length antibodies), multispecific antibodies (e.g. bispecific antibodies), antibody fragments, i.e. antigen binding fragments of an antibody, immunoadhesins and antibody-immunoadhesin chimeras, that specifically recognize (i.e. bind) an antigen. “Antigen binding fragments” comprise a portion of a full-length antibody, preferably the variable domain thereof, or at least the antigen binding site thereof (“an antigen binding fragment of an antibody”). Examples of antigen binding fragments include Fab (fragment antigen binding), scFv (single chain fragment variable), single domain antibodies (nanobodies), diabodies, dsFv, Fab′, diabodies, single-chain antibody molecules, and multispecific antibodies formed from antibody fragments.

The terms “having specificity for”, “specifically binds” or “specific for” with respect to an antigen-binding domain of an antibody, of a fragment thereof or of a CAR refer to an antigen-binding domain which recognizes and binds to a specific antigen, but does not substantially recognize or bind other molecules in a sample. An antigen-binding domain that binds specifically to an antigen from one species may bind also to that antigen from another species. This cross-species reactivity is not contrary to the definition of that antigen-binding domain isspecific. An antigen-binding domain that specifically binds to an antigen may bind also to different allelic forms of the antigen (allelic variants, splice variants, isoforms etc.). This cross reactivity is not contrary to the definition of that antigen-binding domain is specific.

As used herein, the term “antigen” is intended to include substances that bind to or evoke the production of one or more antibodies and may comprise, but is not limited to, proteins, peptides, polypeptides, oligopeptides, lipids, carbohydrates such as dextran, haptens and combinations thereof, for example a glycosylated protein or a glycolipid. The term “antigen” as used herein refers to a molecular entity that may be expressed e.g. on the surface of a target cell and that can be recognized by means of the adaptive immune system including but not restricted to antibodies or TCRs, or engineered molecules including but not restricted to endogenous or transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

The term “soluble antigen” as used herein refers to an antigen that is not immobilized on surfaces such as beads or cell membranes, i.e. it is soluble during the process of binding to a adCAR via a tagged polypeptide as disclosed herein and of subsequently activating the immune cell that expresses said CAR. This definition does not exclude the possibility that the soluble antigen may be immobilized temporary, e.g.by temporary binding to surface of a cell membrane such as a chemokine can do. The soluble antigen is dissolved in a liquid, e.g. in an interstitial fluid of a subject. The soluble antigen as disclosed herein may possess at least an epitope A and an epitope B that can be recognized and bound by antigen binding domains specific for said epitope A and B, respectively. Alternatively, the soluble antigen as disclosed herein may possess the same epitope (epitope E) at least two times, thereby allowing that simultaneously at least two antigen binding domains specific for said epitope (epitope E) bind to the soluble antigen.

The use of the terms “epitope A”, “epitope B”, “epitope C”, “epitope D”, “epitope E” and “epitope F” in the context of a soluble antigen are merely place holder for real epitopes. For example, two times “epitopes F” within a soluble antigen means that an epitope having (comprising) the same primary, secondary or tertiary structure that can be recognized by an antigen binding domain specific for said epitope F are present on the soluble antigen and is available for binding by said antigen binding domain specific for said epitope F. For example, a soluble antigen comprising (having) an epitope A and epitope B means that two different epitopes are present on the soluble antigen that can be recognized and bound by two different antigen binding domains, specific for the epitope A and the epitope B, respectively.

The term “epitope” means the part of an antigen, e.g. a soluble antigen, that may be recognized and specifically bound by antibodies or antigen bindings fragments thereof (antigen binding domains).

The terms “immune cell” or “immune effector cell” may be used interchangeably and refer to a cell that may be part of the immune system and executes a particular effector function such as alpha-beta T cells, NK cells, NKT cells, B cells, innate lymphoid cells (ILC), cytokine induced killer (CIK) cells, lymphokine activated killer (LAK) cells, gamma-delta T cells, regulatory T cells (Treg), monocytes or macrophages. Preferentially these immune cells are human immune cells. Preferred immune cells are cells with cytotoxic effector function such as alpha-beta T cells, NK cells, NKT cells, ILC, CIK cells, LAK cells or gamma-delta T cells. Most preferred immune effector cells are T cells and NK cells. “Effector function” means a specialized function of a cell, e.g. in a T cell an effector function may be cytolytic activity or helper activity including the secretion of cytokines.

Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, preferentially T cell-based or NK cell-based cytotoxic responses to attack cancer cells. T cells that have a natural or genetically engineered reactivity to a patient's cancer are generated in-vitro and then transferred back into the cancer patient. Then the immunotherapy is referred to as “CAR immunotherapy” or in case of use of T cells only as “CAR T cell therapy” or “CAR T cell immunotherapy”.

The term “treatment” as used herein means to reduce the frequency or severity of at least one sign or symptom of a disease.

The terms “therapeutically effective amount” or “therapeutically effective population” mean an amount of a cell population which provides a therapeutic benefit in a subject.

As used herein, the term “subject” refers to an animal. Preferentially, the subject is a mammal such as mouse, rat, cow, pig, goat, chicken dog, monkey or human. More preferentially, the subject is a human. The subject may be a subject suffering from a disease such as cancer (a patient) or from an autoimmune disease or from a allergic disease or from an infectious disease or from graft rejection.

The term “expression” as used herein is defined as the transcription and/or translation of a particular nucleotide sequence driven by its promoter in a cell.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. Especially, the terms refer to the fact that cells, preferentially T cells can be manipulated by recombinant methods well known in the art to express stably or transiently peptides or proteins which are not expressed in these cells in the natural state. For example, T cells, preferentially human T cells are engineered to express an artificial construct such as a chimeric antigen receptor on their cell surface.

The term “cancer” is known medically as a malignant neoplasm. Cancer is a broad group of diseases involving unregulated cell growth and includes all kinds of leukemia. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

The term “autoimmune disease” as used herein is defined as a disorder that results from an autoimmune response. An autoimmune disease is the result of an inappropriate and excessive response to a self-antigen. Examples of autoimmune diseases include but are not limited to, Behcet's disease, Juvenile idiopathic arthritis, Type 1 diabetes, Rheumatoid arthritis, Wegener Granulomatosis, Systemic lupus erythematosus, Systemic sclerosis, Crohn's disease, Graves' disease, Hashimoto thyreoiditis, Goodpasture syndrome, pernicieuse anemia, Primary biliary cholangitis, Myasthenia gravis, Dermato polymyositis, Vasculitis, Mixed connective tissue disease, and Scleroderma.

The term “effector” as used herein refers to a molecule expressed or synthesized by the immune cell as disclosed herein triggered by an activated adapterCAR (anti-tag CAR) as disclosed herein that has bound to the soluble antigen as disclosed herein, wherein said effector has an effect on the effector expressing immune cell itself or on the environment of the effector expressing immune cell when expressed and secreted, wherein said effect is not present when the effector is not expressed and/or synthesized in said immune cell. If the effector has an effect on the immune cell expressing said effector, then the effector may exert its effect intracellularly or alternatively, the effector may be secreted by said immune cell and exerts its effect on said immune cell extracellularly.

The effector may be an antibody or antigen binding fragment thereof, a therapeutic peptide or protein, a cytokine, a chemokine, a receptor, a transcription factor, a siRNA, or shRNA.

The effector may be an antibody or antigen binding fragment thereof. Said antibody or antigen binding fragment thereof may be secreted after triggering the adCAR (and thereby activating the immune cell expressing said adCAR). Said antibody or antigen binding fragment may be a therapeutic or nontherapeutic antibody or antigen binding fragment thereof.

Said antibody or antigen binding fragment thereof may be e.g. prevent angiogenesis, is directed against TAA, interacts with the patients innate immune cells, influences checkpoint inhibition, functions as an agonist, functions as an antagonist or has a neutralizing effect.

Said antigen binding fragments of an antibody may be a e.g. Fab, a scFv or a nanobody.

The effector may be a peptide or protein. Said peptide or protein may be secreted. Said peptide may be a therapeutic or nontherapeutic peptide. Said protein may be a therapeutic or nontherapeutic protein.

Said peptide or protein may be selected e.g. from the group consisting of antithrombins, fibrinolytic, enzymes, antineoplastic agents, hormones, immunosuppressive agents and extra cellular matrix remodeling enzymes.

Said peptide or protein may induce a therapeutic effect e.g. replace a protein that is deficient or abnormal, augment an existing pathway, providing a novel function or activity, interfere with a molecule or organism, deliver other compounds or proteins, protect against a deleterious foreign agent, treat an autoimmune disease or treat cancer by inducing the formation of pores on the target cell, penetrates the target cell or targets TAA to induce cytotoxicity.

The effector may be a cytokine. Said cytokine may be secreted.

Said cytokine may allow local and dose-controlled shaping of the microenvironment that activated the CAR of the immune cell. Said cytokine may be pro- or anti-inflammatory.

Said cytokine may be a Interleukin, a Interferon, a TNF, a TGF-beta or miscellaneous hematopoietins.

Said cytokine may be IL-1α, IL-1β, IL-1RA, IL-18, IL-2, IL-4, IL-7, IL-9, IL-13, IL-15, IL-3, IL-5, GM-CSF, IL-6, IL-11, G-CSF, IL-12, LIF, OSM, IL-10, IL-20, IL-14, IL-16, or IL-17.

Said interferon may be IFN-α, IFN-β or IFN-γ.

Said TNF may be CD154, LT-β, TNF-α, TNF-β, 4-1BBL, APRIL, CD70, CD153, CD178, GITRL, LIGHT, OX40L, TALL-1, TRAIL, TWEAK or TRANCE.

Said TGF-β may be TGF-β1, TGF-β2 or TGF-β3.

Said Miscellaneous hematopoietin may be Epo, Tpo, Flt-3L, SCF, M-CSF or MSP.

The effector may be a chemokine. Said chemokine may be secreted.

Said chemokine may allow time and dose-controlled shaping of the microenvironment that activated the CAR of the immune cell. Said chemokine may be pro- or anti-inflammatory.

Said chemokine may be a C chemokine, a CC chemokine, a CXC chemokine or a CX3C chemokine.

Said C chemokine may be XCL1 or XCL2.

Said CC chemokine may be CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27.

Said CXC chemokine may be CXCL1, CXCL2, CXCL3, CXCL5, CXCL6, CXCL7, CXCL8, CXCL9, CXCL10, CXCL11, CXCL12, CXCL13, CXCL14.

Said CX3C chemokine may be CX3CL1.

The effector may be a receptor. Said receptor may be expressed on the surface of the effector expressing immune cell.

Said receptor may be a Syn Notch receptor. Said Syn Notch receptor may induce a transgene.

Said receptor may be a CAR. Said CAR may have cytotoxicity against a target cell.

Said receptor may be an immunomodulatory receptor. Said immunomodulatory receptor may induce local stimulation or inhibition of immune cells, e.g. B cells, T cells and monocytes.

Said receptor may be a Toll-like receptor.

Said receptor may be a cell adhesion/migration molecule. Said cell adhesion/migration molecule may improve tissue penetration or modulation of immune cell homing.

Said effector may be a transcription factor or a transcription factor associated molecule. Said transcription factor or a transcription factor associated molecule may improve immune cell persistence and proliferation, or may induce transgene expression.

Said transcription factor or a transcription factor associated molecule may be BCL-2, BcL-XL, c-Jun, LMO1, LMO2, BFAT.

The effector may be a RNA. Said RNA may enable gene knockout or protein knockdowns under specific environmental conditions.

Said RNA may be a guide RNA for CRISPR/Cas9, a siRNA or a shRNA.

The terms “nucleic acid” or “polynucleotide” as used interchangeably herein refer to polymers of nucleotides. Polynucleotides, which can be hydrolyzed into monomeric “nucleotides.” The monomeric nucleotides can be hydrolyzed into nucleosides. As used herein, the term “polynucleotides” encompasses, but is not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “promoter” as used herein refers to a DNA sequence recognized by the synthetic machinery of the cell, or introduced synthetic machinery, required to initiate the transcription of a specific polynucleotide sequence.

As used herein, the term “promoter/regulatory sequence” means a nucleic acid sequence which is required for transcription of a gene product operably linked to the promoter/regulatory sequence. In some instances, this sequence may be the core promoter sequence and in other instances, this sequence may also include an enhancer sequence and other regulatory elements which are required for transcription of the gene product. The promoter/regulatory sequence may, for example, be one which expresses the gene product in a tissue-specific manner.

The term “minimal promoter (PMIN)” as used herein refers to the smallest genetic element that is able to induce transcription of a gene located downstream of said minimal promoter. Eukaryotic promoters of protein-coding genes have one or more of three conserved sequences in this region (i.e. the TATA-box, initiator region, and downstream promoter element). A minimal promoter enables low basal leakiness in the absence of specific transcriptional activators and high expression when transcription activators are bound upstream of minimal promoter at their specific DNA binding sites. Alternative minimal promoters can be used, such as minimal TATA box promoter, minimal CMV promoter or minimal IL-2 promoter.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only in the presence or absence of certain conditions such as, for example, when an inducer (e.g. drug, metal ions, alcohol, oxygen, etc.) which corresponds to the promoter is present in the cell. The inducer may be the activation of the intracellular signaling domain of a CAR.

Constitutive promotors that are operatively linked to a transgene, herein e.g. a synthetic transcription factor or an effector such as a second anti-tag CAR, may be for example EF-1 alpha promoter or any other constitutive promoter that drives constitutive expression in immune cells (such as MSCV, PGK-1, UBC, CMV, CAGG, SV40 or pan-hematopoietic promoter, such as vav).

The inducible promoter that is operably linked to the polynucleotide encoding e.g. a synthetic transcription factor or an effector may be any promoter the activation of which is responsive to a transcriptional factor that is increased when immune cells are specifically activated or localized to a given microenvironment (could be e.g. a tumor microenvironment), such as an SP1, BATF, AP-1, IRF4, RUNX, NFAT, NF-κB, STAT5 or STAT3 sensing promoter and a minimal promoter operably linked to an inducible enhancer as described e.g. in WO2019199689A1. The inducible promoter further may comprise a minimal promoter operably linked to an effector. NFAT-inducible promoter can be exchanged with any inducible promoter that specifically binds specific transcriptional factors. Without wishing to be bound by theory, if NFAT responsive element is present, it needs a signal from TCR/CAR or other immune receptors that induces NFAT signaling.

The inducible promoter may be linked to a minimal promoter (PMIN). Alternative minimal promoters can be used, such as minimal TATA box promoter, minimal CMV promoter or minimal IL-2 promoter. In some embodiments, the minimal promoter may be optimized for a desired level or rate of transcription.

In a specific variant, the inducible promoter may be a drug-inducible promoter.

Such a system may comprise a nucleic acid comprising a promoter inducible by a drug. By utilizing a drug-inducible promoter, a transgene expression can be turned on and off in order to avoid toxic side effects and/or to allow the cells to rest during remission. Many of these systems use chimeric transcriptional regulators (e.g. synthetic transcription factors) built from xenogeneic components, thus introducing the complication of immunogenicity when applying these systems to human therapeutics.

In one variant the inducible promoter may be inducible by a drug, i.e. a drug-inducible promoter. The drug is selected based on safety record, favorable pharmacokinetic profile, tissue distribution, a low partition coefficient between the extracellular space and cytosol, low immunogenicity, low toxicities, and/or high expression in lymphocytes. In some alternatives, the inducible promoter is activated by a transcriptional activator (e.g. a synthetic transcription factor) that interacts with a drug. The transcriptional activator is activated or able to bind to and activate the inducible promoter in the presence of the drug. A specific alternative of a drug is a drug that binds to an estrogen receptor ligand binding domain of a transcriptional activator. In some alternatives, the drug includes tamoxifen, its metabolites, analogs, and pharmaceutically acceptable salts and/or hydrates or solvates thereof.

The term “synthetic transcription factor” as used herein may comprise a DNA-binding domain, a drug inducible domain (a drug binding domain) and an effector (activation) domain, that are linked and/or fused whereby the individual domains can be arranged in any order

A DNA binding domain of a synthetic transcription factor may be a protein or a portion of a protein that specifically recognize the DNA binding motif of the drug-inducible promoter and mediate the binding of the synthetic transcription factor to this DNA sequence. Besides zinc finger proteins, TALE (transcription activator-like effector) and Cas9 (Clustered Regulatory Interspaced Short Palindromic Repeats-associated system) may be engineered to recognize a specific DNA sequence.

DNA binding motifs of drug-inducible promoters are specific DNA sequences that are directly or indirectly (in case of Cas9) recognized by the DNA-binding domain of the synthetic transcription factor. E.g. each zinc finger domain specifically recognizes a DNA sequence of 3 bp, thus a three-finger zinc finger protein can be designed to recognize a 9 bp sequence.

Drug-binding domain of a synthetic transcription factor refers to a protein or a portion of a protein that binds to a drug. Upon drug binding, the drug-binding domain enables the transition from an inactive to an active synthetic transcription factor. Examples of drug binding domains are nuclear receptors, extracellular domains of receptors, antigen/substance binding proteins (also dimerizers) and/or active sites of enzymes.

Said DNA binding domain may be e.g. a zinc finger protein (or the DNA binding domain thereof) or a protein comprising or consisting of a POU domain.

An activation domain of a synthetic transcription factor refers to a protein or a portion of a protein that autonomously facilitates the recruitment of the transcriptional machinery to initiate mRNA transcription. Examples of activation domains are VP16, VP64, NFkB p65 and combinations thereof.

E.g. the synthetic transcription factor may comprise a zinc finger protein, the estrogen receptor (ER) and an activation domain., and wherein said drug may be tamoxifen or a tamoxifen metabolite. Said activation domain may be e.g. herpes virus simplex protein VP16, the tetrameric repeat of VP16's minimal activation domain VP64, or the p65 domain of the human endogenous transcription factor NFκB. Said tamoxifen metabolite may be endoxifen or 4-OHT. Said ER may be a ER having point mutations such as murine ER (G525R) or (G521R), human ER (G400V, M543A, L540A) or human ER (G400V, M543A, L544A).

The drug-inducible promoter may be a hybrid promoter comprising a DNA binding motif for said DNA binding domain of the synthetic transcription factor and a minimal promoter.

Said drug-inducible promoter may be a hybrid promoter comprising a zinc finger binding motif and a minimal promoter that comprises a minimal promoter selected from the group consisting of E1b, TK, IL2, CMV, SV40.

### EXAMPLES

### Example 1

### Sensing of Soluble Antigens with Anti-Tag CAR T Cells

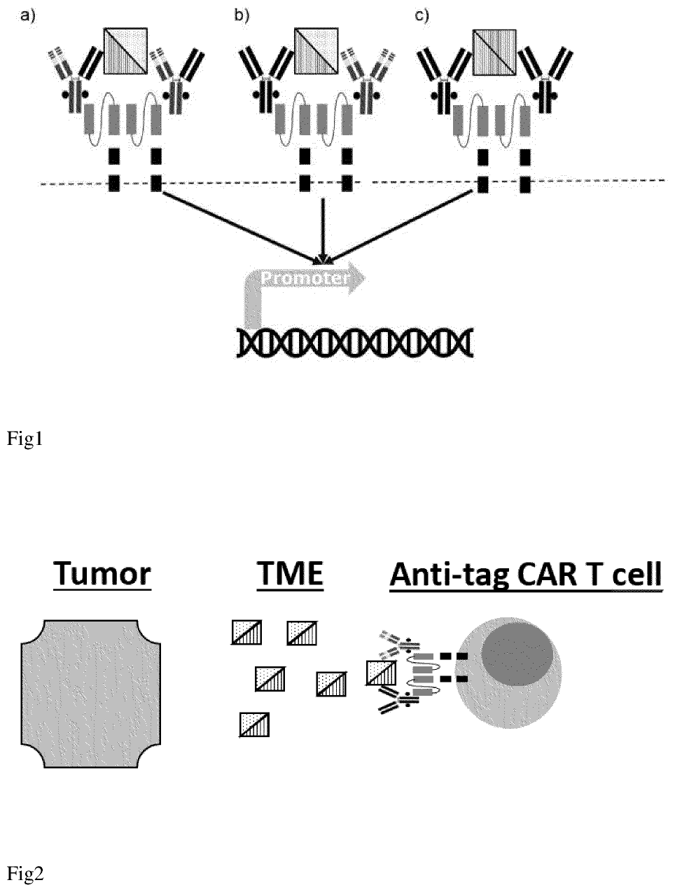

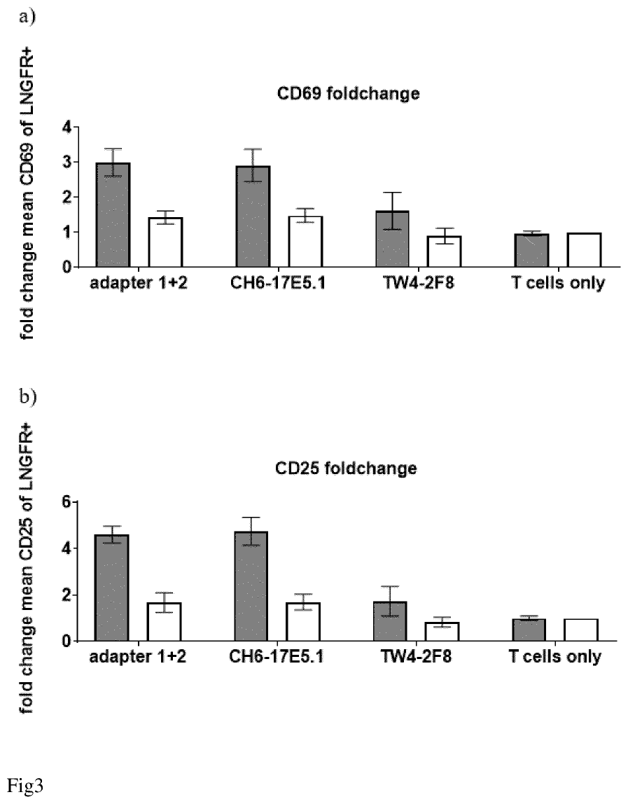

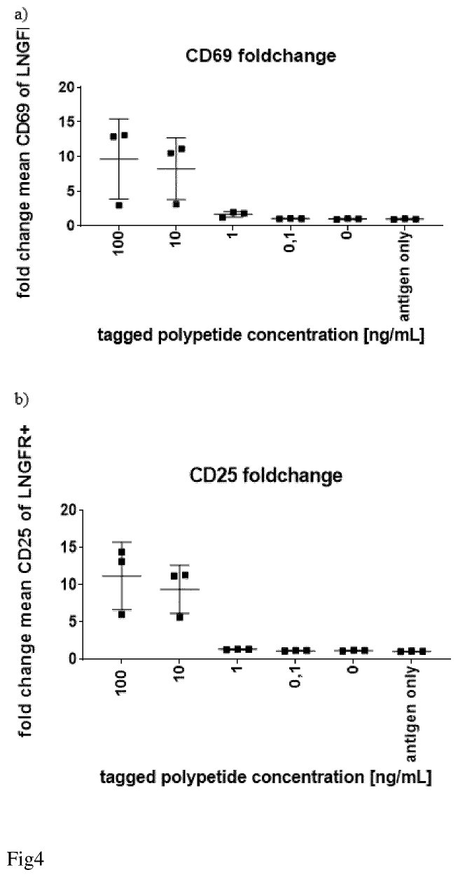

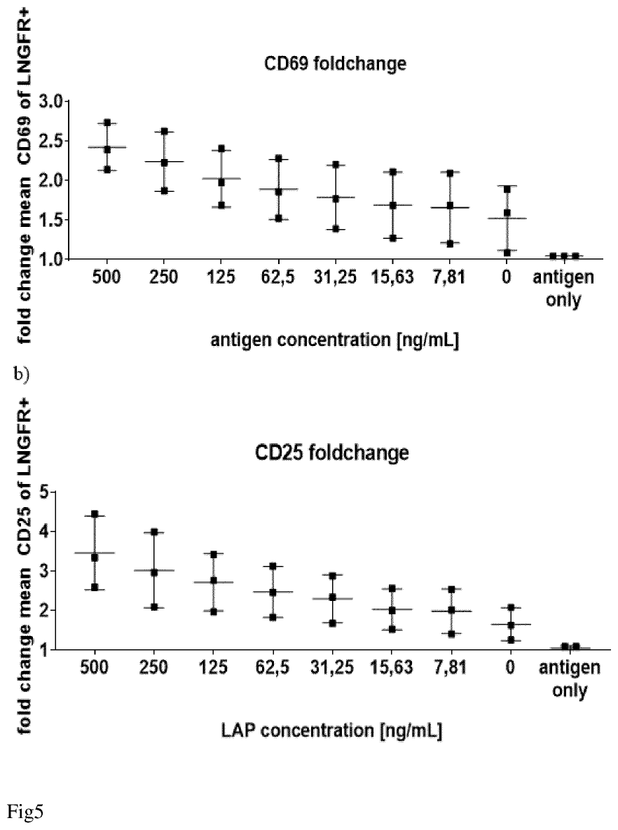

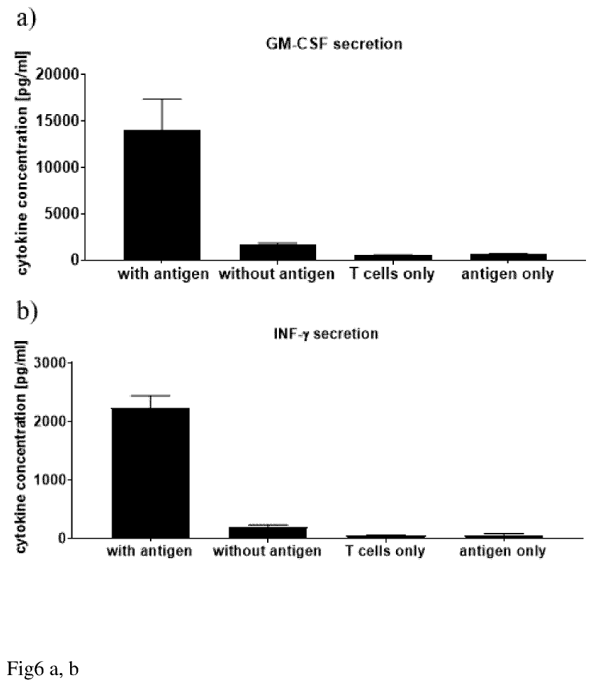

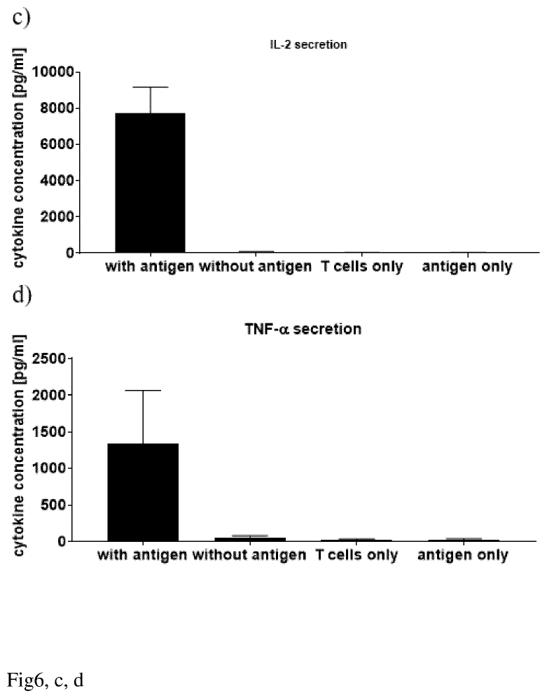

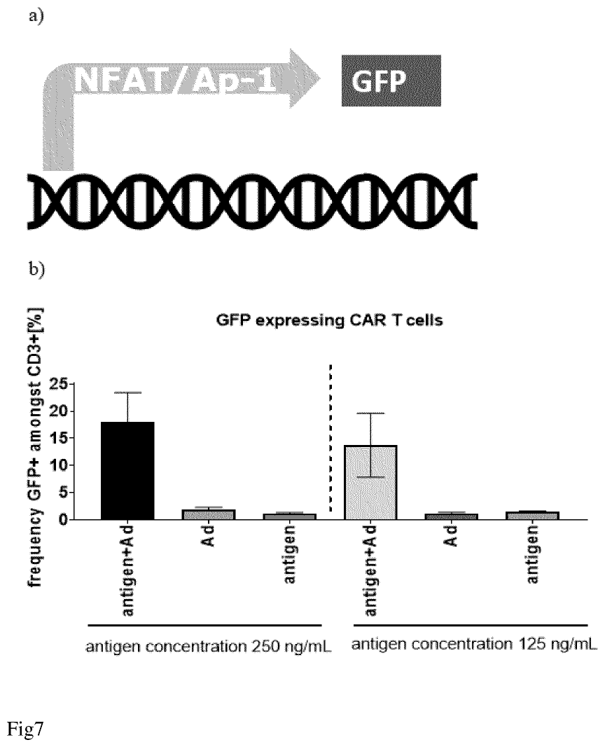

Both the tagged polypeptide and the soluble target antigen are in solution. The anti-tag CAR T cells are in a non-activated status. The binding of the tagged polypeptide to the soluble target and the subsequent binding of the tagged polypetide antigen complex by the anti-tag CAR T cell results in receptor multimerization on the anti-tag CAR T cell surface and activation of T cell intrinsic signaling pathways. The soluble antigen can provide an epitope A and B and the tagged polypeptide has an antigen binding domain for epitope A and B (FIG. 1a) to induce activation. The soluble antigen can provide an epitope A and B wherein the first tagged polypeptide has an antigen binding domain for epitope A and the second tagged polypeptide has an antigen binding domain for epitope B (FIG. 1b) and both tagged polypeptides are needed to induce activation. If the soluble antigen provides at least two times an epitope E one tagged polypeptide with an antigen binding domain for epitope E is needed (FIG. 1c).

### Example 2

### Generation of Anti-Tag CAR T Cells

2.1 Construct Design

Anti-tag CAR T cells contained an anti-biotin scFv as binding moiety. The scFv was linked to human CD8 transmembrane domain via hIgG4 hinge domain. The signaling domain was either composed of 4-1BB and CD3ζ (construct 1) or CD28, 4-1BB and CD3ζ (construct 2). A furin P2A site followed by a truncated LNGFR was 3′ of the CAR construct. The LNGFR was used as a transduction marker.

2.2 Generation of LV Particles and Titration

Lentiviral vector particles were manufactured via transient transfection of HEK-293T cells. The lentiviral vector particles were pseudotyped with VSV-G. For transfection HEK-293T cells were seeded in T175 culture flasks in DMEM (Biowest) supplemented with 2 mM L-Glutamine (Lonza) and 10% FCS (Biochrom) 3 days prior to transfection. At the day of transfection the culture medium was removed and replaced by DMEM (Biowest) supplemented with 2 mM L-Glutamine (Lonza). The cells were transfected with a three plasmid system encoding for VSV-G, gag/pol/rev and the psi positive transfer vector (anti-tag CAR or NFAT-AP1 inducible GFP). After 48 h the supernatant was collected and centrifuged for 10 min at 1000 rpm to remove cellular debris. In addition the supernatant was filtrated trough a 0.45 μm filter. The pellet was re-suspended in ice cold PBS and stored at −80° C.

A functional titer of VSV-G pseudotyped lentiviral vector particles was determined via titration on Sup-T1 cells. 2E5 cells were seeded in 150 μL RPMI (Biowest) supplemented with 2 mM L-Glutamine (Lonza) in 96 well round bottom plates. For transduction 50 μL of serial diluted lentiviral vector particles were added to the seeded cells. 90 μL RPMI (Biowest) supplemented with 2 mM L-Glutamine (Lonza) and 10% FCS (Biochrom) was added after 24 h. The frequency of transduced cells was quantified after 96 h by flow cytometry using a LNGFR APC conjugate (Miltenyi Biotec). Based on the frequency of LNGFR positive cells, the number of seeded cells and the volume of lentiviral particle used for transduction, the titer was calculated. The titer was expressed in transducing units per mL.

2.3 Transduction, Cultivation and Analysis of Anti-Tag CAR T Cells

Anti-tag CAR T cells were manufactured using primary T cells from healthy donors. T cells were isolated from PBMC with the PAN T cell isolation Kit (Miltenyi Biotec) according to the manufactures protocol. Prior to transduction 2E6 T cells were seeded in a 24 well plate with 2 mL TexMACS medium (Miltenyi Biotec) supplemented with IL-7 (Miltenyi Biotec), IL-15 (Miltenyi Biotec) and TransAct (Miltenyi Biotec). After 24 h T cells were transduced with an MOI of 5 by adding the corresponding volume of lentiviral vector particles. On day 3 post activation the culture medium was removed and replaced by TexMACS medium (Miltenyi Biotec) supplemented with IL-7 (Miltenyi Biotec) and IL-15 (Miltenyi Biotec). Frequency of anti-tag CAR positive T cells was analyzed on day 6 after transduction via flow cytometry using a LNGFR APC or LNGFR PE conjugate (Miltenyi Biotec). Transduced T cells were enriched for LNGFR positive cells on day 7 post transduction using MACSelect LNGFR MicroBeads (Miltenyi Biotec). Enrichment procedure was done according to suppliers protocol. Anti-tag CAR T cells were used for functional assays on day 10 after activation or stored in liquid nitrogen.

### Example 3

### Generation of Tagged Polypeptides

In order to generate tagged polypeptides the polypeptide was labeled with biotin. A molecular weight cut off column (Merck Millipore) was equilibrated with 0.1 M NaHCO₃. The molecular weight cut-off was chosen depending on the molecular weight of the used polypeptide. The polypeptide was mixed with one column volume of NaHCO₃ and applied to the column. The solution was centrifuged according to manufactures protocol of the column for 5 min. Next the membrane was rinsed with the supernatant and the protein concentration was determined via absorption measurement at 280 nm. Depending on the coupling rate of the polypeptide the amount of EZ-Link™ NHS-LC-LC-Biotin (Thermo Fisher Scientific, 567.7 g/mol) needed for the coupling reaction was calculated. In order to prepare the tag solution EZ-Link™ NHS-LC-LC-Biotin (Thermo Fisher Scientific, 567.7 g/mol) was dissolved in water free DMSO (Sigma Aldrich) with a final concentration of 10 mg/mL. EZ-Link™ NHS-LC-LC-Biotin (Thermo Fisher Scientific, 567.7 g/mol) and polypeptide were mixed at the desired ratio and incubated at 21° C. for 60 min. Next the reaction mix was applied to a equilibrated molecular weight cut off column (Merck Millipore) and washed with four column volumes PBS. The protein concentration of the final product was analyzed via absorption at 280 nM. The coupling rate was analyzed via mass spectrometry.

### Example 4

### Analyzing the Activation of Anti-Tag CAR T Cells with Soluble Antigens via Analysis of Activation Markers

Lentiviral particles encoding for the anti-tag CAR were manufactured as described in example 2.2 Generation of LV particles and titration. On day 10 5E4 CAR positive T cells (construct 2) manufactured as described in example 2 Generation of anti-tag CAR T cells were co-cultured with soluble antigen in a total volume of 200 μL TexMACS (Miltenyi Biotec) in 96-well plate. As a soluble antigen, recombinant human LAP (R&D Systems) was reconstituted in PBS according to supplier's instructions. As polypeptides, anti-human LAP antibody clone1: CH6-17E5.1 (Miltenyi Biotec) and clone2: TW4-2F8 (BioLegend) were modified as described in Example 3 Generation of tagged polypeptides. The activation of anti-tag CAR T cells was analyzed by titrating either the concentration of the tagged polypeptides: 100 ng/mL-0.1 ng/mL of each polypeptide and fixing the concentration of the soluble antigen: 250 ng/mL (FIG. 4) or by titrating the soluble antigen: 500 ng/mL-7.81 ng/mL and fixing the concentration of the tagged polypeptides: 100 ng/mL (FIG. 5). The co-culture was incubated over night at 37° C. and 5% CO₂. Activation was quantified via flow cytometric analysis of surface expression of CD69 and CD25. CD69 expression was quantified using a CD69 VioBlue REA824 (Miltenyi Biotec) conjugate. CD25 expression was quantified using a CD25PE-Vio770 REA570 (Miltenyi Biotec) conjugate.

### Example 5

### Analyzing the Activation of Anti-Tag CAR T Cells with Soluble Antigens via Cytokine Release Assay

To analyze the cytokine secretion of anti-tag CART cells after stimulation with soluble antigens a co-culture was setup as. Lentiviral particles encoding for the anti-tag CAR were manufactured as described in example 2.2 Generation of LV particles and titration. Primary T cells were genetically modified and analyzed as described in example 2 Generation of anti-tag CAR T cells. On day 10 5E4 anti-tag CAR positive cells (construct 1) were seeded in a 96 well flat bottom plate in a total assay volume of 200 μL. Anti-tag CAR T cells were co-cultured with 125 ng/mL soluble LAP (R&D Systems) and 10 ng/mL of tagged polypeptide 1: antibody clone CH6-17E5.1 (Miltenyi Biotec) and tagged polypeptide 2: antibody clone clone TW4-2F8 (Biolegend). Cytokines were analyzed in the supernatant (FIG. 6). Therefore, 100 μL of supernatant were analyzed with human MACS Plex 12 cytokine kit (Miltenyi Biotec) according to manufactures protocol after 24 h.

### Example 6

### Analyzing the Activation of CAR T Cells with Soluble Antigens via Analysis of the Expression of a NFAT/AP-1 Controlled Trans Gene

As described in example 2.2 Generation of LV particles and titration, LV was produced coding for GFP downstream of a human NFAT-AP-1 binding site. This construct was used as a fluorescent reporter for T cell activation and transgene expression in anti-tag CAR T cells (construct 1). T cells were co-transduced as described in in example 2 Generation of anti-tag CAR T cells with the NFAT/AP-1 GFP construct and an anti-tag CAR. On day 10 5E4 anti-tag CAR positive cells were seeded in a 96 well flat bottom plate in a total assay volume of 200 μL. Anti-tag CAR T cells were co cultured with 250 ng/mL and 125 ng/mL soluble LAP (R&D Systems) and 10 ng/mL of a tagged anti LAP polypeptide clone: CH6-17E5.1 (Miltenyi Biotec). The induction of transgene expression was analyzed by flow cytometry after 4 days (GFP excitation 488 nm laser, band pass filter 525/50 nm) (FIG. 7)

### Example 7

### Analyzing the Activation of CAR T Cells with Oligomeric Soluble Antigens via Analysis of Activation Markers and Cytokine Secretion

Lentiviral particles encoding for the anti-tag CAR were manufactured as described in example 2.2 Generation of LV particles and titration. Primary T cells were genetically modified and analyzed as described in example 2 Generation of anti-tag CAR T cells. On day 10 5E4 anti-tag CAR positive cells (construct 1) were seeded in a 96 well flat bottom plate. Anti-tag CAR T cells were co-cultured with 125 ng/mL soluble LAP (R&D Systems) and 10 ng/mL of tagged polypeptide 1: antibody clone CH6-17E5.1 (Miltenyi Biotec) and tagged polypeptide 2: antibody clone clone TW4-2F8 (Biolegend), or with a 1:1 mixture of tagged polypeptide 1 and tagged polypeptide 2. The cells and all reagents were diluted in TexMACS (Miltenyi Biotec). The total assay volume was 200 μL. The co-culture was incubated over night at 37° C. and 5% CO₂. Activation was quantified via flow cytometric analysis of surface expression of CD69 and CD25 (FIG. 3). CD69 expression was quantified using a CD69 VioBlue REA824 (Miltenyi Biotec) conjugate. CD25 expression was quantified using a CD25PE-Vio770 REA570 (Miltenyi Biotec) conjugate.

1.-14. (canceled)

15. A combination of pharmaceutical agents for administration to a subject in need thereof, the combination comprising the following components:
a) an immune cell comprising a polynucleotide that contains a nucleic acid sequence encoding a chimeric antigen receptor (CAR) that comprises:
i) an antigen binding domain specific for a tag that is present on one or more tagged polypeptides,
ii) a transmembrane domain,
iii) an intracellular signaling domain; and
b) said one or more tagged polypeptides, which together contain antigen binding domains specific for one or more epitopes on a soluble antigen that is endogenous to subject;
wherein said soluble antigen endogenous to the subject has sufficient copies of said one or more epitopes such that binding of the tagged polypeptide(s) to the soluble antigen and binding of the immune cell to the tagged polypeptide(s) will result in activation of an effector function by the immune cell.

16. The combination of claim 15, wherein component (b) comprises a tagged polypeptide that contains an antigen binding site specific for epitope A and an antigen binding site specific for epitope B;
wherein the soluble antigen in the subject contains both epitope A and epitope B.

17. The combination of claim 15, wherein component (b) comprises two different tagged polypeptides having the same tag,
wherein the first tagged polypeptide contains an antigen binding site specific for epitope A,
wherein the second tagged polypeptide contains an antigen binding site specific for epitope B;
wherein the soluble antigen in the subject contains both epitope A and epitope B.

18. The combination of claim 15, wherein component (b) comprises a tagged polypeptide that contains multiple antigen binding sites specific for epitope E,
wherein the soluble antigen in the subject contains at least two copies of epitope E.

19. The combination according to claim 15, wherein said intracellular signaling domain of the CAR includes at least one primary cytoplasmic signaling domain comprising an immunoreceptor tyrosine-based activation motif (ITAM).

20. The combination according to claim 15, wherein the soluble antigen that is endogenous to the subject is selected from the following:
a soluble antigen of a tumor microenvironment (TME) of a subject who has cancer;
a soluble antigen specifically associated with an autoimmune disease in a subject;
a soluble antigen specifically associated with an allergic disease in a subject;
a soluble antigen specifically associated with an infectious disease in a subject; and
a soluble antigen specifically associated with rejection of a graft in a subject.

21. The combination according to claim 15, further comprising:
c) one or more additional tagged polypeptides, which together contain an antigen binding domain specific for one or more epitopes on a second soluble antigen that is endogenous to the subject;
wherein component (b) and component (c) both have the same tag.

22. The combination according to claim 21, wherein component (c) comprises a tagged polypeptide that contains an antigen binding site specific for epitope C and an antigen binding site specific for epitope D;
wherein the second soluble antigen in the subject contains both epitope C and epitope D.

23. The combination according to claim 21, wherein component (c) comprises two different tagged polypeptides having the same tag,
wherein the first tagged polypeptide contains an antigen binding site specific for epitope C,
wherein the second tagged polypeptide contains an antigen binding site specific for epitope D;
wherein the second soluble antigen in the subject contains both epitope C and epitope D.

24. The combination according to claim 21, wherein component (c) comprises a tagged polypeptide that contains multiple antigen binding sites specific for epitope F,
wherein the second soluble antigen contains at least two copies of epitope F.

25. The combination according to claim 21, wherein:
if the endogenous soluble antigen is an antigen of a tumor microenvironment (TME) of a subject who has cancer, then the second soluble antigen is another endogenous antigen of the TME in the subject;
if the endogenous soluble antigen is an antigen specifically associated with an autoimmune disease in the subject, then the second soluble antigen is another endogenous antigen specifically associated with said autoimmune disease,
if the endogenous soluble antigen is an antigen specifically associated with an allergic disease in a subject, then the second soluble antigen is another endogenous antigen specifically associated with said allergic disease, and
if the endogenous soluble antigen is an antigen specifically associated with an infectious disease in a subject, then the second soluble antigen is another endogenous antigen specifically associated with said infectious disease.

26. The combination according to claim 15, wherein the polynucleotide in the immune cell further comprises:
(iv) a constitutive promoter operably linked to the nucleic acid sequence encoding the CAR, and
(v) an inducible promoter operably linked to a nucleic acid sequence encoding an effector.

27. The combination according to claim 26, wherein said inducible promoter drives expression of the effector when the antigen binding domain of the CAR binds to the tagged polypeptide(s) bound to said soluble antigen.

28. The combination according to claim 15, wherein polynucleotide in the immune cell further comprises:
iv) a constitutive promoter operably linked to the nucleic acid sequence encoding the CAR,
v) an inducible promoter operably linked to a nucleic acid sequence encoding a synthetic transcription factor for a drug-inducible promoter, wherein said synthetic transcription factor comprises a DNA binding domain, a drug-binding domain and an activation domain, wherein said synthetic transcription factor is activated by binding to said drug, and
vi) a drug-inducible promotor operably linked to a nucleic acid sequence encoding an effector.

29. The combination according to claim 28,
wherein said inducible promoter is selected from an SP1, BATF, AP-1, IRF4, RUNX, NFAT, NF-kB, STAT5 and a STAT3-sensing promotor, and
wherein said drug-inducible promoter is a hybrid promoter comprising a DNA binding motif for said DNA binding domain and a minimal promoter.

30. The combination according to claim 15, wherein the immune cell and the tagged polypeptide are contained in a single pharmaceutical composition.

31. The combination according to claim 15, wherein the immune cell and the tagged polypeptide are contained in separate pharmaceutical compositions for sequential administration to the subject.

32. The combination according to claim 15, wherein the immune cell is a T cell, and the effector function is cytolytic activity, helper activity, or cytokine secretion.

33. The combination according to claim 15, wherein the immune cell is a T cell or an NK cell, and the effector function is a cytotoxic attack on cancer cells.

34. A complex formed in situ following administration of the combination of claim 15 to a subject in need thereof, the complex comprising:
1) a soluble antigen that is endogenous to the subject;
2) the one or more tagged polypeptides from said combination bound to the soluble antigen; and
3) the immune cell from said combination bound to the one or more tagged polypeptides.

35. A method of activating an effector function of an immune cell at the site of an endogenous soluble antigen in the subject, the method comprising contacting the soluble antigen with components (a) and (b) of the combination of claim 15.

36. A method for treating a subject in need thereof with an immune cell that expresses a chimeric antigen receptor (CAR), the method comprising administering to the subject simultaneously or sequentially the following pharmaceutical agents:
a) said immune cell, wherein the CAR expressed thereby contains:
i) an antigen binding domain specific for a tag on one or more tagged polypeptides,
ii) a transmembrane domain,
iii) an intracellular signaling domain; and
b) said one or more tagged polypeptides, which together contain antigen binding domains specific for one or more epitopes on a soluble antigen that is endogenous to the subject;
wherein said soluble antigen endogenous to the subject has sufficient copies of said one or more epitopes such that binding of the tagged polypeptide(s) to the soluble antigen and binding of the immune cell to the tagged polypeptide(s) will result in activation of an effector function by the immune cell in the subject, thereby treating the subject.

37. The method of claim 36, wherein pharmaceutical agent (b) comprises a tagged polypeptide that contains an antigen binding site specific for epitope A and an antigen binding site specific for epitope B;
wherein the soluble antigen in the subject contains both epitope A and epitope B.

38. The method of claim 36, wherein component (b) comprises two different tagged polypeptides having the same tag,
wherein the first tagged polypeptide contains an antigen binding site specific for epitope A,
wherein the second tagged polypeptide contains an antigen binding site specific for epitope B;
wherein the soluble antigen in the subject contains both epitope A and epitope B.

39. The method of claim 36, wherein pharmaceutical agent (b) comprises a tagged polypeptide that contains multiple antigen binding sites specific for epitope E,
wherein the soluble antigen in the subject contains at least two copies of epitope E.

40. The method of claim 36, wherein the immune cell and the tagged polypeptide are administered to the subject together as part of a single composition.

41. The method of claim 36, wherein the subject has cancer.

42. The method of claim 36, wherein the subject has an autoimmune condition, an allergic condition, or an infectious condition, or wherein the subject is experiencing rejection of a graft.
